(19) United States
(12) Patent Application Publication
(10) Pub. No.: US 2022/0395498 A1
(43) Pub. Date: Dec. 15, 2022

(54) TARGETED THERAPEUTICS

(71) Applicant: TVA (ABC), LLC, Wilmington, DE (US)

(72) Inventors: Leila Alland, Bernardsville, NJ (US); Christopher Sears, Belmont, MA (US); Rajesh R. Shinde, Lexington, MA (US); Beata Sweryda-Krawiec, Marlborough, MA (US); Tsun P. Au Yeung, Arlington, MA (US); Mark T. Bilodeau, Waltham, MA (US); Sudhakar Kadiyala, Newton, MA (US); Eugene Zhorov, Marblehead, MA (US); Richard Wooster, Natick, MA (US)

(21) Appl. No.: 17/877,514

(22) Filed: Jul. 29, 2022

Related U.S. Application Data
(63) Continuation of application No. 16/625,358, filed on Dec. 20, 2019, now abandoned.
(60) Provisional application No. 62/642,154, filed on Mar. 13, 2018.
(60) Provisional application No. 62/522,316, filed on Jun. 20, 2017.

(51) Int. Cl. A61K 31/4745; A61K 47/26; A61K 9/00

(52) U.S. Cl. CPC A61K 31/4745; A61K 47/26; A61K 9/0019

(57) ABSTRACT

The present invention provides pharmacological compounds including an effector moiety conjugated to a binding moiety that directs the effector moiety to a biological target of interest. Likewise, the present invention provides compositions, kits, and methods (e.g., therapeutic, diagnostic, and imaging) including the compounds. The compounds can be described as a protein interacting binding moiety-drug conjugate (SDC-TRAP) compounds, which include a protein interacting binding moiety and an effector moiety. For example, in certain embodiments directed to treating cancer, the SDC-TRAP can include an Hsp90 inhibitor conjugated to a cytotoxic agent as the effector moiety.

### CROSS REFERENCE TO RELATED APPLICATIONS

This application is a continuation application of U.S. application Ser. No. 16/625,358 filed Dec. 20, 2019, entitled “TARGETED THERAPEUTICS,” which is a 35 U.S.C. § 371 U.S. National Stage Entry of International Application No. PCT/US2018/038174 filed Jun. 19, 2018, entitled “TARGETED THERAPEUTICS,” which claims priority to U.S. Provisional Application No. 62/522,316, filed on Jun. 20, 2017, entitled “TARGETED THERAPEUTICS,” and U.S. Provisional Application No. 62/642,154, filed on Mar. 13, 2018, entitled “TARGETED THERAPEUTICS,” the contents of each of which are incorporated herein by reference in their entirety.

### FIELD OF THE DISCLOSURE

The present invention relates to pharmacological compounds including an effector moiety conjugated to a binding moiety that directs the effector moiety to a biological target of interest. The compounds have broad pharmacological applications, including therapeutics, diagnostics, and imaging. For example, the compounds can specifically direct therapeutic effector moieties to target cells or tissue of interest, for targeted chemotherapeutic treatment of conditions such as cancer.

### BACKGROUND

Although tremendous advances have been made in chemotherapy, currently available therapeutics and therapies remain unsatisfactory and the prognosis for the majority of patients diagnosed with chemotherapeutically treated diseases (e.g., cancer) remains poor. Often, the applicability and/or effectiveness of chemotherapy, as well as other therapies and diagnostics employing potentially toxic moieties, is limited by undesired side effects.

Many disease and disorders are characterized by the presence of high levels of certain proteins in specific types of cells. In some cases, the presence of these high levels of protein is caused by overexpression. Historically, some of these proteins have been useful targets for therapeutic molecules or used as biomarkers for the detection of disease. One class of overexpressed intracellular protein that has been recognized as a useful therapeutic target is known as the heat shock proteins.

Heat shock proteins (HSPs) are a class of proteins that are up-regulated in response to elevated temperature and other environmental stresses, such as ultraviolet light, nutrient deprivation, and oxygen deprivation. HSPs have many known functions, including acting as chaperones to other cellular proteins (called client proteins) to facilitate their proper folding and repair, and to aid in the refolding of misfolded client proteins. There are several known families of HSPs, each having its own set of client proteins. Hsp90 is one of the most abundant HSP families, accounting for about 1-2% of proteins in a cell that is not under stress and increasing to about 4-6% in a cell under stress.

Inhibition of Hsp90 results in degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Hsp90 has been shown by mutational analysis to be necessary for the survival of normal eukaryotic cells. However, Hsp90 is overexpressed in many tumor types, indicating that it may play a significant role in the survival of cancer cells and that cancer cells may be more sensitive to inhibition of Hsp90 than normal cells. For example, cancer cells typically have a large number of mutated and overexpressed oncoproteins that are dependent on Hsp90 for folding. In addition, because the environment of a tumor is typically hostile due to hypoxia, nutrient deprivation, acidosis, etc., tumor cells may be especially dependent on Hsp90 for survival. Moreover, inhibition of Hsp90 causes simultaneous inhibition of a number of oncoproteins, as well as hormone receptors and transcription factors, making it an attractive target for an anti-cancer agent. In view of the above, Hsp90 has been an attractive target of drug development, including such Hsp90 inhibitor (Hsp90i) compounds as ganetespib, AUY-922, and IPI-504. At the same time, the advancement of certain of these compounds which showed early promise, e.g., geldanamycin, has been slowed by those compounds' toxicity profile. Hsp90i compounds developed to date are believed to show great promise as cancer drugs, but other ways the ubiquity of Hsp90 in cancer cells might be leveraged have heretofore remained unexplored until now. Accordingly, the need exists for therapeutic molecules that selectively target proteins, such as Hsp90, that are overexpressed in cells associated with particular diseases or disorders.

### SUMMARY OF THE DISCLOSURE

The present invention provides pharmacological molecules (“SDC-TRAPs”) including an effector moiety conjugated to a binding moiety, which directs the effector moiety into a target cell of interest in a manner that traps the molecule in the target cell. Methods of making and using the SDC-TRAPs are also provided.

The present invention is described in further detail by the figures and examples below, which are used only for illustration purposes and are not limiting.

Other features and advantages of the instant invention will be apparent from the following detailed description and claims.

### DETAILED DESCRIPTION

The present invention provides molecules including an effector moiety conjugated to a binding moiety that directs the effector moiety to a biological target of interest. The molecules of the invention allow for selective targeting of an effector moiety by trapping the molecules of the invention in a desired cell, e.g., a cancer cell. The molecules can be described as Small molecule Drug Conjugates that are TRAPped intracellularly (SDC-TRAP), due to their selective binding to high concentration intracellular proteins. In order for the molecules of the invention to be trapped within the cells of interest, the binding moieties that are part of the SDC-TRAP molecules interact with proteins that are overexpressed in targeted cells. In exemplary embodiments, the proteins that are overexpressed are characteristic of a particular disease or disorder. Accordingly, the present invention provides compositions, kits, and methods (e.g., therapeutic, diagnostic, and imaging) that include the molecules of the invention.

In one embodiment of the invention, SDC-TRAPs allow for the delivery of an effector molecule that would otherwise be unsuitable for administration alone due to toxicity and/or undesired systemic effects. Using the targeted delivery molecules described herein (SDC-TRAPs) allows for effector moieties that are too toxic to administer by current methods to be dosed at lower levels thereby allowing the toxic effector to be targeted to specific diseased cells at sub-toxic levels.

In various exemplary aspects and embodiments, the present invention provides compounds for treating cancer. For example, an SDC-TRAP can comprise an Hsp90 binding moiety (i.e., targeting Hsp90, which is overexpressed in cancer cells compared to normal cells) and an effector moiety (e.g., the Hsp90 binding moiety can be an Hsp90 inhibitor that is conjugated to a cytotoxic agent). As indicated above, the invention is exemplified herein in terms of Hsp90-targeted binding moieties and cytotoxic agents. Other binding moieties that are contemplated, mentioned or described herein are intended to be included within the scope of the invention.

In various aspects and embodiments, the present invention provides an SDC-TRAP comprising a binding moiety and an effector moiety, wherein the SDC-TRAP molecule is able to enter a cell by passive transport. The ability of an SDC-TRAP to enter a cell by passive transport can be a result of one or more unique chemical properties of the SDC-TRAP (e.g., size, weight, charge, polarity, hydrophobicity, etc.) and can facilitate the delivery and/or action of the SDC-TRAP. The ability of an SDC-TRAP to enter a cell by passive transport is a functional property, which along with its physico-chemical properties, differentiates SDC-TRAPs from other targeted molecules such as antibody-drug conjugates.

In various aspects and embodiments, the present invention provides an SDC-TRAP comprising a binding moiety and an effector moiety, wherein SDC-TRAP molecule is able to enter a cell by active transport. The ability of an SDC-TRAP to enter a cell by active transport can be a result of one or more unique chemical properties of the SDC-TRAP and can facilitate the delivery and/or action of the SDC-TRAP. Example of SDC-TRAP active transport can include, for example, endocytosis, phagocytosis, pinocytosis, and exocytosis.

In various aspects and embodiments, the present invention provides an SDC-TRAP having a molecular weight of less than about 5000 Daltons (e.g., less than about 5000, 2500, 2000, 1600, 1550, 1500, 1450, 1400, 1350, 1300, 1250, 1200, 1150, 1100, 1050, 1000, 950, 900, 850, 800, 750, 700, 650, 600, 550, 500, 450, 400, 350, 300, 250, 200, etc.). Similarly, in various aspects and embodiments, the present invention provides a binding moiety having a molecular weight of less than about 2500 Dalton (e.g., less than about 2500, 2000, 1600, 800, 750, 700, 650, 600, 550, 500, 450, 400, 350, 300, 250, 200, 150, 100, etc.) and/or an effector moiety having a molecular weight of less than about 2500 Dalton (e.g., less than about 2500, 2000, 1600, 800, 750, 700, 650, 600, 550, 500, 450, 400, 350, 300, 250, 200, 150, 100, etc.). The overall molecular weight of an SDC-TRAP, and the individual weights of a binding moiety, effector moiety, and any linking moiety, can affect transport of the SDC-TRAP. In various examples, it has been observed that lower molecular weights can facilitate delivery and/or activity of an SDC-TRAP.

In various aspects and embodiments, the present invention provides an SDC-TRAP comprising an Hsp90 binding moiety and an effector moiety, wherein the Hsp90 binding moiety and the effector moiety are approximately equal in size (e.g., the Hsp90 binding moiety and the effector moiety have less than about a 25, 50, 75, 100, 125, 150, 175, 200, 225, 250, 275, 300, 325, 350, 375, 400, etc. Dalton difference in molecular weight.) In various examples, it has been observed that lower differences in molecular weight can facilitate delivery and/or activity of an SDC-TRAP.

In various aspects and embodiments, the present invention provides an SDC-TRAP comprising a target protein-interacting binding moiety. A target protein-interacting binding moiety can selectively interact with any one or more domains of a target protein. For example, where a target protein is Hsp90, the binding moiety can be an Hsp90 binding moiety that interacts with the N-terminal domain of Hsp90, the C-terminal domain of Hsp90, and/or the middle domain of Hsp90. Selective interaction with any one or more domains of a target protein can advantageously increase specificity and/or increase the concentration of molecular targets within a target tissue and/or cell.

In various aspects and embodiments, the present invention provides an SDC-TRAP comprising a binding moiety having a high affinity for a molecular target (e.g., a K_{d} of 50, 100, 150, 200, 250, 300, 350, 400 nM or higher). For example, where a binding moiety is an Hsp90 binding moiety, the Hsp90 binding moiety can have a K_{d} of 50, 100, 150, 200, 250, 300, 350, 400 nM or higher. A binding moiety having a high affinity for a molecular target can advantageously improve targeting and/or increase the resonance time of the SDC-TRAP in a target cell and/or tissue.

In various aspects and embodiments, the present invention provides an SDC-TRAP comprising a binding moiety (e.g., Hsp90 binding moiety) and an effector moiety, wherein when administered to a subject the SDC-TRAP is present at a ratio of about 2:1 in tumor cells compared to plasma. The ratio can be higher, for example, about 5:1, 10:1, 25:1, 50:1, 75:1, 100:1, 150:1, 200:1, 250:1, 300:1, 400:1, 500:1, 600:1, 700:1, 800:1, 900:1, 1000:1, or greater. In various aspects and embodiments, the ratio is at 1, 2, 3, 4, 5, 6, 7, 8, 12, 24, 48, 72, or more hours from administration. The effectiveness of targeting can be reflected in the ratio of SDC-TRAP in a target cell and/or tissue compared to plasma.

In various aspects and embodiments, the present invention provides an SDC-TRAP comprising a binding moiety (e.g., Hsp90 binding moiety) and an effector moiety, wherein the SDC-TRAP is present in target (e.g., cancer) cells for at least 24 hours. The SDC-TRAP can be present in cancer cells for longer, for example, for at least 48, 72, 96, or 120 hours. It can be advantageous for an SDC-TRAP to be present in target cells for longer periods of time to increase the therapeutic effect of a given dose of SDC-TRAP and/or increase an interval between administrations of SDC-TRAP.

In various aspects and embodiments, the present invention provides an SDC-TRAP comprising a binding moiety (e.g., Hsp90 binding moiety) and an effector moiety, wherein the effector moiety is released for a period of at least 6 hours. The effector moiety can be released for a longer period, for example, for at least 12, 24, 48, 72, 96, or 120 hours. Selective release can be used to control, delay, and/or extend the period of release of an effector moiety and, therefore, increase the therapeutic effect of a given dose of SDC-TRAP, decrease the undesired side effects of a given dose of SDC-TRAP, and/or increase an interval between administrations of SDC-TRAP.

In various aspects and embodiments, the present invention provides an SDC-TRAP comprising an Hsp90 binding moiety and an effector moiety, wherein the effector moiety is selectively released inside a target (e.g., cancer) cell. Selective release can be achieved, for example, by a cleavable linker (e.g., an enzymatically cleavable linker). Selective release can be used to decrease undesired toxicity and/or unwanted side effects. For example, an SDC-TRAP can be designed where an effector moiety such is inactive (or relatively inactive) in a conjugated form, but active (or more active) after it is selectively released inside a target (e.g., cancer) cell.

In various aspects and embodiments, the present invention provides an SDC-TRAP comprising a binding moiety (e.g., Hsp90 binding moiety) and an effector moiety, wherein the SDC-TRAP allows for the use of an effector moiety that is otherwise toxic or unfit for administration to a subject. The effector moiety can be unfit for administration to a subject because of undesired toxicity. In such cases, a strategy such as selective release may be used to address the undesired toxicity. The effector moiety can be unfit for administration to a subject because of undesired targeting or a lack of targeting. Targeting can address such problems, for example, by minimizing systemic toxicity while maximizing local toxicity at a target (e.g., a tumor).

In various aspects and embodiments, the present invention provides an SDC-TRAP comprising a binding moiety (e.g., Hsp90 binding moiety) and an effector moiety, wherein the binding moiety is an inhibitor (e.g., Hsp90 inhibitor) that is ineffective as a therapeutic agent when administered alone. In such cases, the SDC-TRAP may facilitate an additive or synergistic effect between the binding moiety and effector moiety, thereby advantageously improving the efficacy and/or reducing the side effects of a therapy.

In order that the present invention may be more readily understood, certain terms are first defined. In addition, it should be noted that whenever a value or range of values of a parameter are recited, it is intended that values and ranges intermediate to the recited values are also intended to be part of this invention. Unless defined otherwise, all technical and scientific terms used herein have the same meaning as commonly understood to one of ordinary skill in the art to which this invention belongs. It is also to be understood that the terminology employed is for the purpose of describing particular embodiments, and is not intended to be limiting.

### Definitions

The articles “a,” “an,” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature.

As used herein, the term “benefit” refers to something that is advantageous or good, or an advantage. Similarly, the term “benefiting,” as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2− amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. Samples are typically removed from the subject prior to analysis. However, tumor samples can be analyzed in the subject, for example, using imaging or other detection methods.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level in a control sample that consists of a group of measurements may be determined, e.g., based on any appropriate statistical measure, such as, for example, measures of central tendency including average, median, or modal values.

As used herein, the term “obtaining” is understood herein as manufacturing, purchasing, or otherwise coming into possession of.

As used herein, the term “identical” or “identity” is used herein in relation to amino acid or nucleic acid sequences refers to any gene or protein sequence that bears at least 30% identity, more preferably 40%, 50%, 60%, 70%, 75%, 80%, 81%, 82%, 83%, 84%, 85%, 86%, 87%, 88%, 89%, 90%, 91%, 92%, 93%, 94%, and most preferably 95%, 96%, 97%, 98%, 99% or more identity to a known gene or protein sequence over the length of the comparison sequence. Protein or nucleic acid sequences with high levels of identity throughout the sequence can be said to be homologous. A “homologous” protein can also have at least one biological activity of the comparison protein. In general, for proteins, the length of comparison sequences will be at least 10 amino acids, preferably 10, 20, 30, 40, 50, 60, 70, 80, 90, 100, 150, 175, 200, 250, or at least 300 amino acids or more. For nucleic acids, the length of comparison sequences will generally be at least 25, 50, 100, 125, 150, 200, 250, 300, 350, 400, 450, 500, 550, 600, 650, 700, 800, or at least 850 nucleotides or more.

As used herein, “detecting,” “detection” and the like are understood that an assay performed for identification of a specific analyte in a sample. The amount of analyte or activity detected in the sample can be none or below the level of detection of the assay or method.

The terms “modulate” or “modulation” refer to upregulation (i.e., activation or stimulation), downregulation (i.e., inhibition or suppression) of a level, or the two in combination or apart. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, “level of activity” is understood as the amount of protein activity, typically enzymatic activity, as determined by a quantitative, semi-quantitative, or qualitative assay. Activity is typically determined by monitoring the amount of product produced in an assay using a substrate that produces a readily detectable product, e.g., colored product, fluorescent product, or radioactive product.

As used herein, “changed as compared to a control” sample or subject is understood as having a level of the analyte or diagnostic or therapeutic indicator (e.g., marker) to be detected at a level that is statistically different than a sample from a normal, untreated, or control sample control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection the amount and measurement of the change can vary. Changed as compared to a control reference sample can also include a change in one or more signs or symptoms associated with or diagnostic of disease, e.g., cancer. Determination of statistical significance is within the ability of those skilled in the art, e.g., the number of standard deviations from the mean that constitute a positive result.

“Elevated” or “lower” refers to a patient's value of a marker relative to the upper limit of normal (“ULN”) or the lower limit of normal (“LLN”) which are based on historical normal control samples. As the level of the marker present in the subject will be a result of the disease, and not a result of treatment, typically a control sample obtained from the patient prior to onset of the disease will not likely be available. Because different labs may have different absolute results, values are presented relative to that lab's upper limit of normal value (ULN).

The “normal” level of expression of a marker is the level of expression of the marker in cells of a subject or patient not afflicted with cancer. In one embodiment, a “normal” level of expression refers to the level of expression of the marker under normoxic conditions.

An “over-expression” or “high level of expression” of a marker refers to an expression level in a test sample that is greater than the standard error of the assay employed to assess expression, and is preferably at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of the marker in a control sample (e.g., sample from a healthy subject not having the marker associated disease, i.e., cancer). In one embodiment, expression of a marker is compared to an average expression level of the marker in several control samples.

A “low level of expression” or “under-expression” of a marker refers to an expression level in a test sample that is less than at least 0.9, 0.8, 0.7, 0.6, 0.5, 0.4, 0.3, 0.2, or 0.1 times the expression level of the marker in a control sample (e.g., sample from a healthy subject not having the marker associated disease, i.e., cancer). In one embodiment, expression of a marker is compared to an average expression level of the marker in several control samples.

As used herein, “binding” is understood as having at least a 10² or more, 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

The terms “administer,” “administering” or “administration” can include any method of delivery of a pharmaceutical composition or agent into a subject's system or to a particular region in or on a subject. In certain embodiments of the invention, an Hsp90 inhibitor is administered intravenously, intramuscularly, subcutaneously, intradermally, intranasally, orally, transcutaneously, or mucosally. In a preferred embodiment, an agent is administered intravenously. Administering can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “high concentration” refers to the concentration of SDC-TRAP that accumulates in target cells of the invention due to the selective binding of the binding moiety of the SDC-TRAP to the target protein. In one embodiment, the concentration is higher than in similar cells that do not overexpress the target protein, e.g., lung cancer cells as compared to non-cancerous lung cells. In another embodiment, the concentration is higher in target cells compared to cells that do not express, or overexpress, the target protein. In exemplary embodiments, the high concentration is 1.5, 2, 3, 4, 5, 10, 15, 20, 50, 100, 1000 times or more than cells that are not targeted by the SDC-TRAP molecules of the invention.

The term “moiety” refers generally to a portion of a molecule, which may be a functional group, a set of functional groups, and/or a specific group of atoms within a molecule, that is responsible for a characteristic chemical, biological, and/or medicinal property of the molecule.

The term “binding moiety” refers to low molecular weight (e.g., less than about 2500, 200, 1600, 800, 700, 600, 500, 400, 300, 200, or 100 etc. Dalton) organic compounds, which may serve as a therapeutic or a regulator of a biological process. Binding moieties include molecules that can bind to a biopolymer such as protein, nucleic acid, or polysaccharide and acts as an effector, altering the activity or function of the biopolymer. Binding moieties can have a variety of biological functions, serving as cell signaling molecules, as tools in molecular biology, as drugs in medicine, as pesticides in farming, and in many other roles. These compounds can be natural (such as secondary metabolites) or artificial (such as antiviral drugs); they may have a beneficial effect against a disease (such as drugs) or may be detrimental (such as teratogens and carcinogens). Biopolymers such as nucleic acids, proteins, and polysaccharides (such as starch or cellulose) are not binding moieties, although their constituent monomers—ribo- or deoxyribo-nucleotides, amino acids, and monosaccharides, respectively—are often considered to be. Small oligomers are also usually considered binding moieties, such as dinucleotides, peptides such as the antioxidant glutathione, and disaccharides such as sucrose.

As used herein, a “protein interacting binding moiety” or “binding moiety” refers to a binding moiety, or portion thereof, that interacts with a predetermined target. The interaction is achieved through some degree of specificity and/or affinity for the target. Both specificity and affinity is generally desirable, although in certain cases higher specificity may compensate for lower affinity and higher affinity may compensate for lower specificity. Affinity and specificity requirements will vary depending upon various factors including, but not limited to, absolute concentration of the target, relative concentration of the target (e.g., in cancer vs. normal cells), potency and toxicity, route of administration, and/or diffusion or transport into a target cell. The target can be a molecule of interest and/or localized in an area of interest. For example, the target can be a therapeutic target and/or localized in an area targeted for a therapy (e.g., a protein that is overexpressed in cancerous cells, as compared to normal cells). In one particular example, a target can be a chaperonin protein such as Hsp90 and the binding moiety can be an Hsp90 binding moiety (e.g., therapeutic, cytotoxic, or imaging moiety). Preferentially, the binding moiety will enhance, be compatible with, or not substantially reduce, passive transport of a conjugate including the binding moiety into a cell, e.g., a cell comprising a target protein.

The term “effector moiety” refers to a molecule, or portion thereof, that has an effect on a target and/or proximally to the target. In various preferred embodiments, the effector moiety is a binding moiety, or portion thereof. An effect can include, but is not limited to, a therapeutic effect, an imaging effect, and/or a cytotoxic effect. At a molecular or cellular level, an effect can include, but is not limited to, promotion or inhibition of the target's activity, labeling of the target, and/or cell death. Preferentially, the effector moiety will enhance, be compatible with, or not substantially reduce, passive transport of a conjugate including the effector moiety into a cell comprising a target. Different effector moieties can be used together and therapeutics in accordance with the present invention may include more than one effector moiety (e.g., two or more different (or same) effector moieties in a single therapeutic in accordance with the present invention, two or more different therapeutics in accordance with the present invention including different effector moieties).

In some embodiments, the effector moiety is selected from the group consisting of peptidyl-prolyl isomerase ligands; rapamycin, cyclosporin A; steroid hormone receptor ligands, antimitotic agents, actin binding agents, camptothecins, topotecan, combretastatins, capecitabine, gemcitabine, vinca alkaloids, platinum-containing compounds, metformin, HDAC inhibitors, thymidylate synthase inhibitors; nitrogen mustards; 5-fluorouracil (5-FU) and its derivatives, or a combination thereof.

In some embodiments, the effector moiety is selected from the group consisting of FK506; rapamycin, cyclosporin A, estrogen, progestin, testosterone, taxanes, colchicine, colcemid, nocadozole, vinblastine, vincristine, cytochalasin, latrunculin, phalloidin, lenalidomide, pomalidomide, SN-38, topotecan, combretastatins, capecitabine, gemcitabine, vinca alkaloids, metformin, suberoylanilidehydroxamic acid (SAHA), methotrexate, pemetrexed, raltitrexed, bendamustine, melphalan; 5-fluorouracil (5-FU), vedotin and DM1, or a combination thereof.

The term “small molecule drug conjugate that is trapped intracellularly” or “binding moiety drug conjugate that is trapped intracellularly” or “SDC-TRAP” refers to a binding moiety and effector moiety joined to one another, or acting as if joined to one another. A binding moiety and effector moiety can be joined through essentially any chemical or physical force, either directly (e.g., binding moiety and effector moiety viewed as two moieties on the same molecule, or a single moiety having both functions) or through an intermediate (e.g., linker). For example, a binding moiety and effector moiety can be joined by one or more covalent bonds, ionic bonds, hydrogen bonds, the hydrophobic effect, dipole-dipole forces, ion-dipole forces, dipole-induced dipole forces, instantaneous dipole-induced dipole forces, and/or combinations thereof. Preferentially, the SDC-TRAP will be capable of passive and/or active transport into a cell comprising a target. Moreover, SDC-TRAP molecules of the invention may comprise multiple effector molecules conjugated to the binding moiety.

The term “linker” or “linking moiety,” as used herein in the context of binding moiety, effector moieties, and/or SDC-TRAPs refers to a chemical moiety that joins two other moieties (e.g., a binding moiety and an effector moiety). A linker can covalently join a binding moiety and an effector moiety. A linker can include a cleavable linker, for example an enzymatically cleavable linker. A linker can include a disulfide, carbamate, amide, ester, and/or ether linkers.

As used herein, a “ligand” is a substance (e.g., a binding moiety) that can form a complex with a biomolecule. The ligand and/or formation of the ligand-biomolecule complex can have a biological or chemical effect, such as a therapeutic effect, cytotoxic effect, and/or imaging effect.

As used herein, a “prodrug” is a pharmacological substance that is administered in an inactive or less than fully active form and that is subsequently converted to an active pharmacological agent (i.e., the drug) through a metabolic processes. Prodrugs can be used to improve how the intended drug is absorbed, distributed, metabolized, and/or excreted. A prodrug may also be used to improve how selectively the intended drug interacts with cells or processes that are not its intended target (e.g., to reduce adverse or unintended effects of the intended drug, for example a chemotherapy drug).

The phrase “Hsp90 ligand or a prodrug thereof” refers generally to molecules that bind to and in some cases effect Hsp90, and inactive forms (i.e., prodrugs) thereof. An Hsp90 ligand can be an “Hsp90 inhibitor,” which is understood as a therapeutic agent that reduces the activity of Hsp90 either by directly interacting with Hsp90 or by, for example, preventing the formation of the Hsp90/CDC37 complex such that the expression and proper folding of at least one client protein of Hsp90 is inhibited. “Hsp90” includes each member of the family of heat shock proteins having a mass of about 90-kilodaltons. For example, in humans the highly conserved Hsp90 family includes cytosolic Hsp90^{α} and Hsp90^{β} isoforms, as well as GRP94, which is found in the endoplasmic reticulum, and HSP75/TRAP1, which is found in the mitochondrial matrix. As used herein, Hsp90 inhibitors include, but are not limited to ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIIB-021, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, WC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i, herbinmycin A, radicicol, CCT018059, PU-H71, or celastrol.

The term “therapeutic moiety” refers to molecule, compound, or fragment thereof that is used for the treatment of a disease or for improving the well-being of an organism or that otherwise exhibit healing power (e.g., pharmaceuticals, drugs, and the like). A therapeutic moiety can be a chemical, or fragment thereof, of natural or synthetic origin used for its specific action against disease, for example cancer. Therapeutic agents used for treating cancer may be called chemotherapeutic agents. As described herein, a therapeutic moiety is preferentially a small molecule. Exemplary small molecule therapeutics include those that are less than 800 Daltons, 700 Daltons, 600 Daltons, 500 Daltons, 400 Daltons, or 300 Daltons.

The term “cytotoxic moiety” refers to molecule, compound, or fragment thereof that has a toxic or poisonous effect on cells, or that kills cells. Chemotherapy and radiotherapy are forms of cytotoxic therapy. Treating cells with a cytotoxic moiety can produce a variety of results—cells may undergo necrosis, stop actively growing and dividing, or activate a genetic program of controlled cell death (i.e., apoptosis). Examples of cytotoxic moieties include, but are not limited to, SN-38, bendamustine, VDA, doxorubicin, pemetrexed, vorinostat, lenalidomide, irinotecan, ganetespib, docetaxel, 17-AAG, 5-FU, abiraterone, crizotinib, KW-2189, BUMB2, DC1, CC-1065, adozelesin, or fragment(s) thereof.

The term “imaging moiety” refers to a molecule, compound, or fragment thereof that facilitates a technique and/or process used to create images or take measurements of a cell, tissue, and/or organism (or parts or functions thereof) for clinical and/or research purposes. An imaging moiety can produce, for example, a signal through emission and/or interaction with electromagnetic, nuclear, and/or mechanical (e.g., acoustic as in ultrasound) energy. An imaging moiety can be used, for example, in various radiology, nuclear medicine, endoscopy, thermography, photography, spectroscopy, and microscopy methods.

“Pharmaceutical conjugate” refers to a non-naturally occurring molecule that includes a binding moiety (e.g., an Hsp90-targeting moiety) associated with an effector moiety, where these two components may also be covalently bonded to each other either directly or through a linking group.

The term “drug” refers to any active agent that affects any biological process. Active agents that are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g., cell death, cell proliferation etc.

By “pharmacokinetic property” is meant a parameter that describes the disposition of an active agent in an organism or host.

By “half-life” is meant the time for one-half of an administered drug to be eliminated through biological processes, e.g., metabolism, excretion, etc.

The term “efficacy” refers to the effectiveness of a particular active agent for its intended purpose, i.e., the ability of a given active agent to cause its desired pharmacologic effect.

Binding Moiety-Effector Moiety Drug Conjugates that are Trapped Intracellularly (SDC-TRAPs)

The present invention provides SDC-TRAPs, as well as SDC-TRAP compositions, kits, and methods of use thereof. SDC-TRAPs include a binding moiety (e.g., a binding moiety such as a ligand) conjugated to an effector moiety (e.g., a pharmacological agent such as a drug or imaging agent). These two moieties can be joined by a linker, e.g., a covalently-bonded linking group. SDC-TRAPs are useful in a variety of therapeutic, imaging, diagnostic, and/or research applications. In one illustrative example of cancer therapy, an SDC-TRAP can be a pharmaceutical conjugate of an Hsp90-binding moiety such as an Hsp90 ligand or inhibitor associated with an effector moiety such as a therapeutic or cytotoxic agent.

In various embodiments, an SDC-TRAP can be further characterized in that the binding moiety (e.g., targeting moiety) and effector moiety are different, such that the pharmaceutical conjugate may be viewed as a heterodimeric compound produced by the joining of two different moieties. In terms of function, SDC-TRAP molecules have a targeting functionality and effector functionality (e.g., therapeutic, imaging, diagnostic). These functions are provided by corresponding chemical moieties that can be different (or, in some cases, the same). SDC-TRAPs can include any one or more binding moieties conjugated to any one or more effector moieties. In some embodiments, a composition or method can include a combination of two or more binding moeities and/or two or more effector moieties (e.g., a combination therapy and/or multi target therapy) embodied in one or more different types of SDC-TRAPs.

In various embodiments, an SDC-TRAP is further characterized by its ability to passively diffuse and/or be actively transported into a target cell of interest. The diffusion and/or transport properties of the SDC-TRAP can be derived, at least in part, from ionic, polar, and/or hydrophobic properties of the SDC-TRAP. In preferred embodiments, the SDC-TRAP enter cells primarily by passive diffusion. The diffusion and/or transport properties of the SDC-TRAP can be derived, at least in part, from the molecular weight of the SDC-TRAP, the binding moiety, the effector moiety, and/or the similarity in weight between the binding moiety and the effector moiety. SDC-TRAPs are desirably small, such as in comparison to antibody-drug conjugates (“ADCs”). For example, the molecular weight of an SDC-TRAP can be less than about 5000, 2500, 2000, 1600, 1500, 1400, 1300, 1200, 1100, 1000, 900, 800, 700, 600, 500, or 400 Daltons. A binding moiety and an effector moiety can each be less than about 1000, 900, 800, 700, 600, 500, 400, 300, or 200 Daltons. A binding moiety and an effector moiety can be approximately equal in size (e.g., differ in weight by less than 400, 350, 300, 250, 200, 150, 100, or 50 Daltons).

Delivery of an effector molecule by an SDC-TRAP can result in greater potency compared to administering an untargeted drug comprising the same effector moiety, for example, because the SDC-TRAP can be localized at a desired target for an extended period of time through the association of a binding moiety and its target. Such localization can cause an effector moiety to be active and/or released in a target cell and/or tissue over an extended period of time. This resonance time can be selected through deliberate design of a linker moiety. In contrast, administration of the drug by itself in vivo can be more apt to have a shorter resonance time in a given target cell and/or tissue—if it traverses into the cell at all—due to the lack of an “anchor” within the cell.

SDC-TRAPs, in part because they comprise a targeting moiety and are relatively small in size, can be efficiently taken up or internalized by a target cell. Conversely, uptake or internalization is relatively inefficient for ADCs, which must deal with limited antigen expression and relatively inefficient internalization mechanisms for the antibody portion of the molecule. Hsp90 provides a good illustrative example of a difference between SDC-TRAPs and conventional ADCs. By way of comparison, the localization rate of radiolabeled monoclonal antibodies at a tumor in patients is low, on the order of 0.003-0.08% of the injected dose/g tumor. In contrast, a much higher accumulation rate (15-20% injected dose/g tumor) has been measured for SDC-TRAPs in mouse tumor xenografts.

SDC-TRAP pharmaceutical conjugates in accordance with the present invention can represent a significant advance over the state of the art in targeted drugs. SDC-TRAPs have broad application in many therapeutic, imaging, and diagnostic application. As discussed above, SDC-TRAPs are advantageously small in comparison to ADCs, enabling better penetration of solid tumors and more rapid clearance from normal tissues (e.g., reduced toxicity). The design of SDC-TRAPs (e.g., a structure-property relationship) can be established using methods and rationales within the grasp of those of ordinary skill in the art, and companion imaging diagnostics for targeted therapies may also easily be provided, in view of the simpler chemistry involved.

SDC-TRAPs of the invention are characterized by selective targeting of SDC-TRAPs to target cells in which a target protein is overexpressed. This leads to high intracellular concentrations of SDC-TRAP molecules in target cells as compared to non-targeted cells. Likewise, SDC-TRAPs of the invention are characterized by low concentrations of SDC-TRAP in non-targeted cells.

One illustrative embodiment involves a conjugate of an Hsp90 binding moiety linked to a chelator (i.e., the effector moiety, for metals such as In or Gd, which conjugate may function as an imaging agent for the cells/tissues targeted by the conjugate). Another, illustrative embodiment involves a conjugate of an Hsp90 binding moiety linked to a chemotherapeutic (i.e., the effector moiety, for example, SN-38). Alternatively, an illustrative SDC-TRAP is contemplated wherein an Hsp90 targeting moiety bearing radiolabeled halogen (e.g., such as an iodine isotope) can serve to image the cells/tissues targeted by the conjugate, and the effector moiety can be drug to treat the targeted cells/tissues. The progression of treatment may therefore be determined by imaging the tissues being treated and reviewing the images for the presence or absence of the labeled conjugate. Such embodiments are readily adaptable to essentially any cancer, or other chemotherapeutic target. Molecular targets (e.g., interacting with a binding moiety) used to target a particular cell or tissue can be selected based upon their presence in the target cell or tissue and/or their relative abundance in the target cell or tissue (e.g., disease-related versus normal cells).

SDC-TRAP molecules of the present invention represent a new class of drugs. One particular advantage of SDC-TRAPs is that they can be designed to selectively deliver an effector moiety (e.g., a chemotherapeutic drug) into a targeted cell because of the relative overexpression or presence of a binding moiety's molecular target in the cell. After the binding moiety binds the molecular target, the effector moiety is thereafter available (e.g., through cleavage of a linker moiety joining the binding moiety and the effector moiety) to act upon the cell. Accordingly, SDC-TRAPs employ a different mechanism from strategies currently used in the art, for example delivering an Hsp90 inhibitor to a cell using HPMA copolymer-Hsp90i conjugates, Hsp90i prodrugs, nanoparticle-Hsp90i conjugates, or micellar methodologies.

SDC-TRAPs can also be described by the formula:

Binding Moiety-L-E

where “binding moiety” is a protein interacting binding moiety; L is a conjugation or linking moiety (e.g., a bond or a linking group); and E is an effector moiety. These elements are discussed in the context of additional illustrative examples below. However, while features of each element may be discussed separately, design and selection of an SDC-TRAP can involve the interplay and/or cumulative effect of features of each element (e.g., diffusion, binding, and effect).

Once SDC-TRAP molecules of the invention enter a target cell the effector molecule is released from the SDC-TRAP. In one embodiment, the effector molecule has no activity until it is released from the SDC-TRAP. Accordingly, once the SDC-TRAP molecules enter a target cell an equilibrium exists between free and bound SDC-TRAP molecules. In one embodiment, the effector moiety is only released from the SDC-TRAP when the SDC-TRAP is not associated with the target protein. For example, when an SDC-TRAP molecule is not bound intracellular enzymes can access the linker region thereby freeing the effector moiety. Alternatively, when free SDC-TRAP molecules may be able to release effector molecules through, for example, hydrolysis of the bond or linker that connects the binding moiety and effector moiety.

Accordingly, the rate of effector molecule release and the amount of effector molecule released can be controlled by using binding moieties that bind to the target protein with different affinities. For example, binding moieties that bind to the target protein with lower affinity will be free, resulting in higher concentrations of unbound intracellular SDC-TRAP, and thereby resulting in higher concentrations of free effector molecule. Therefore, in at least one embodiment, irreversibly-binding binding moieties are incompatible with certain aspects of the invention, e.g., those embodiments where effector molecule release is based on free intracellular SDC-TRAP molecules.

In one embodiment, SDC-TRAPs have favorable safety profiles, for example, when compared to, for example, the binding moiety or effector molecule alone. One reason for the increased safety profile is the rapid clearance of SDC-TRAP molecules that do not enter into a target cell.

A number of exemplary SDC-TRAP molecules are set forth in the examples. Specifically a number of Hsp90-specific SDC-TRAP molecules are described and used to demonstrate the efficacy of SDC-TRAP molecules.

### Binding Moieties

A primary role of a binding moiety is to ensure that the SDC-TRAP delivers its payload—the effector moiety—to its target by binding to a molecular target in or on a target cell or tissue. In this respect, it is not necessary that the binding moiety also have an effect on the target (e.g., in the case of an Hsp90-targeting moiety, to inhibit Hsp90 in the manner that Hsp90 is are known to do, that is, exhibit pharmacological activity or interfere with its function), but in some embodiments, the binding moiety does have an effect on the target. Accordingly, in various embodiments, an activity of the SDC-TRAP is due solely to the effector moiety exerting a pharmacological effect on the target cell(s), which has been better facilitated by the pharmaceutical conjugate targeting the target cell(s). In other embodiments, an activity of the SDC-TRAP is due in part to the binding moiety—that is, the binding moiety can have an effect beyond targeting.

The molecular target of a binding moiety may or may not be part of a complex or structure of a plurality of biological molecules, e.g., lipids, where the complexes or structures may include lipoproteins, lipid bilayers, and the like. However, in many embodiments, the molecular target to which the binding moiety binds will be free (e.g., cytoplasmic globular protein and/or not be part of a macromolecular assembly or aggregation). The present invention can exploit the selectively high presence of a molecular target in locations of high physiological activity (e.g., Hsp90 in oncological processes). For example, where a drug target is an intracellular drug target, a corresponding molecular target (e.g., Hsp90) can be present in the cell. Likewise, where a drug target is an extracellular drug target, a corresponding molecular target (e.g., Hsp90) can be extracellular, proximal, or associated with the extracellular cell membrane of the target cell or tissue.

In various embodiments, a binding moiety can effect a target cell or tissue (e.g., in the case of an Hsp90-targeting moiety that in fact inhibits Hsp90, for example, Hsp90i). In such embodiments, a pharmacological activity of the binding moiety contributes to, complements, or augments, the pharmacological activity of the effector moiety. Such embodiments go beyond the advantages combination therapies (e.g., a cancer combination therapy of Hsp90i and a second drug such as ganetespib or crizotinib) by providing a therapy that can be carried out by administration of a single SDC-TRAP that realizes both the benefits of the combination therapy and targeting. Other examples of such SDC-TRAPs include conjugates of an Hsp90i (such as ganetespib) and a second cancer drug such as docetaxel or paclitaxel (e.g., in NSCLC); BEZ235 (e.g., in melanoma, prostate and/or NSCLC); temsirolimus (e.g., renal cell carcinoma (RCC), colon, breast and/or NSCLC); PLX4032 (e.g., in melanoma); cisplatin (e.g., colon, breast cancer); AZD8055 (e.g., in NSCLC); and crizotinib (e.g., ALK⁺ NSCLC).

A range of pharmaceutical activities can be achieved by judicious selection of a binding moiety and an effector moiety. For example, for treating solid tumors, e.g., colon cancer, high continuous doses of antimetabolites such as capecitabine or gemcitabine tend to be required in combination with other drugs. A conjugate having an Hsp90-targeting moiety with lower binding affinity or inhibitory activity to Hsp90, e.g., as determined by a HER2 degradation assay, can be designed to meet this need. Such a conjugate can comprise an effector moiety that is a strong, potent antimetabolite such as 5-FU, to afford a high dose of the conjugate that may be dosed relatively frequently. Such an approach not only achieves the aim of providing a high dose of an antimetabolite fragment at the tumor, but also lowers the toxicity of administering the drug on its own, owing to the plasma stability of SDC-TRAPs of the invention, and the ability of the Hsp90-targeting moiety to deliver the antimetabolite to the desired cells or tissues.

In embodiments where solid tumors such as SCLC or colorectal cancer are to be treated with drugs such as topotecan or irinotecan, only low doses of the drug may be dosed. Due to the very high intrinsic activity of these drugs, an SDC-TRAP should be designed to provide a low dose of such drugs at the target tissue. In this scenario, for example, an Hsp90-targeting moiety having a higher binding affinity or inhibitory activity to Hsp90 (e.g., as determined by a HER2 degradation assay) can sufficiently maintain the presence of the drug in the tissue at a very high level, to ensure that enough of the drug reaches and is retained by the desired target tissue due to the low dosing.

In various illustrative embodiments where a molecular target of a binding moiety is Hsp90, the binding moiety can be an Hsp90-targeting moiety, for example a triazole/resorcinol-based compound that binds Hsp90, or a resorcinol amide-based compound that binds Hsp90, e.g., ganetespib or a tautomer/derivative/analog thereof, AUY-922 or a tautomer/derivative/analog thereof, or AT-13387 or a tautomer/derivative/analog thereof.

In another embodiment, the binding moiety may advantageously be an Hsp90-binding compound of formula (I):

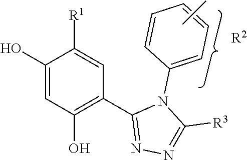

wherein
R¹ may be alkyl, aryl, halide, carboxamide or sulfonamide; R² may be alkyl, cycloalkyl, aryl or heteroaryl, wherein when R² is a 6 membered aryl or heteroaryl, R² is substituted at the 3- and 4-positions relative to the connection point on the triazole ring, through which a linker L is attached; and R³ may be SH, OH, —CONHR⁴, aryl or heteroaryl, wherein when R³ is a 6 membered aryl or heteroaryl, R³ is substituted at the 3 or 4 position.

In another embodiment, the binding moiety may advantageously be an Hsp90-binding compound of formula (II):

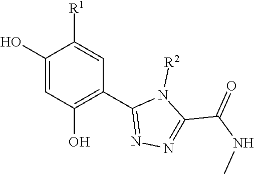

wherein
R¹ may be alkyl, aryl, halo, carboxamido, sulfonamido; and R² may be optionally substituted alkyl, cycloalkyl, aryl or heteroaryl. Examples of such compounds include 5-(2,4-dihydroxy-5-isopropylphenyl)-N-(2-morpholinoethyl)-4-(4-(morpholinomethyl)phenyl)-4H-1,2,4-triazole-3-carboxamide and 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide.

In another embodiment, the binding moiety may advantageously be an Hsp90-binding compound of formula (III):

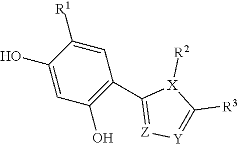

wherein
X, Y, and Z may independently be CH, N, O or S (with appropriate substitutions and satisfying the valency of the corresponding atoms and aromaticity of the ring); R¹ may be alkyl, aryl, halide, carboxamido or sulfonamido; R² may be substituted alkyl, cycloalkyl, aryl or heteroaryl, where a linker L is connected directly or to the extended substitutions on these rings; R³ may be SH, OH, NR⁴R⁵ AND —CONHR⁶, to which an effector moiety may be connected; R⁴ and R⁵ may independently be H, alkyl, aryl, or heteroaryl; and R⁶ may be alkyl, aryl, or heteroaryl, having a minimum of one functional group to which an effector moiety may be connected. Examples of such compounds include AUY-922:

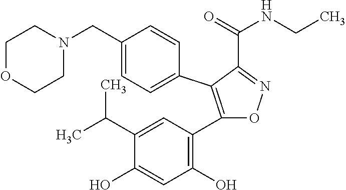 [SMILES: CCNC(=O)c1noc(-c2cc(C(C)C)c(O)cc2O)c1-c1ccc(CN2CCOCC2)cc1]

In another embodiment, the binding moiety may advantageously be an Hsp90-binding compound of formula (IV):

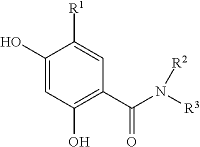

wherein
R¹ may be alkyl, aryl, halo, carboxamido or sulfonamido; R² and R³ are independently C₁-C₅ hydrocarbyl groups optionally substituted with one or more of hydroxy, halogen, C₁-C₂ alkoxy, amino, mono- and di-C₁-C₂ alkylamino; 5- to 12-membered aryl or heteroaryl groups; or, R² and R³, taken together with the nitrogen atom to which they are attached, form a 4- to 8-membered monocyclic heterocyclic group, of which up to 5 ring members are selected from O, N and S. Examples of such compounds include AT-13387:

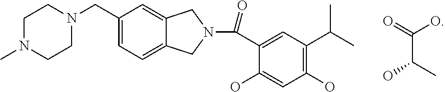

In certain embodiments, to enhance the bioavailability or delivery of the pharmaceutical conjugate, the binding moiety may be a prodrug of the Hsp90-binding compound.

Specific examples of suitable Hsp90-targeting moieties include

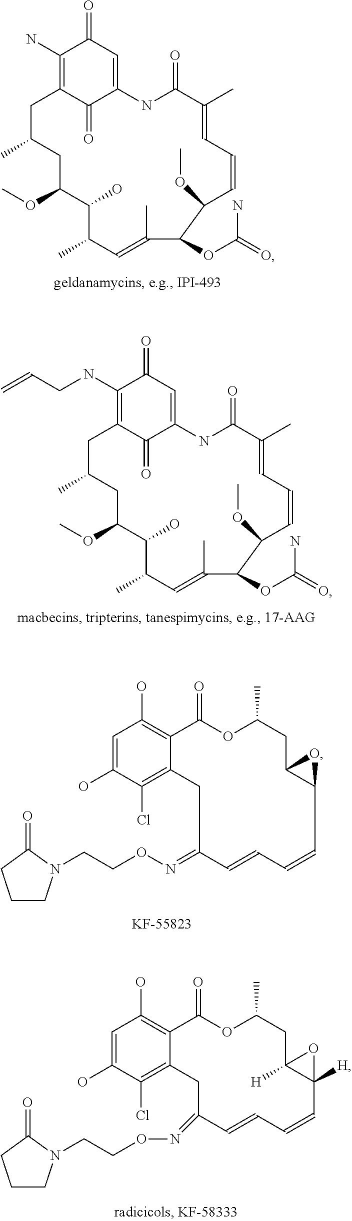

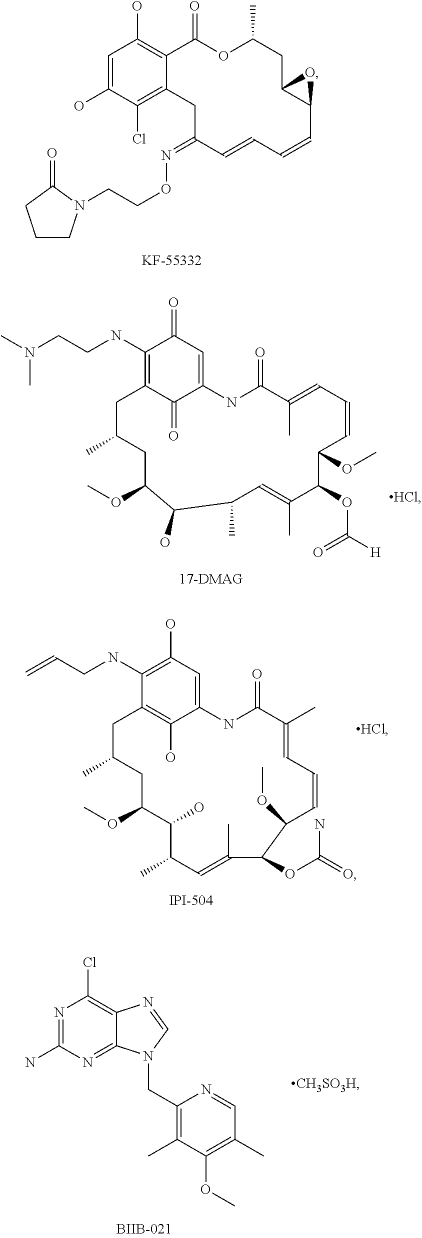

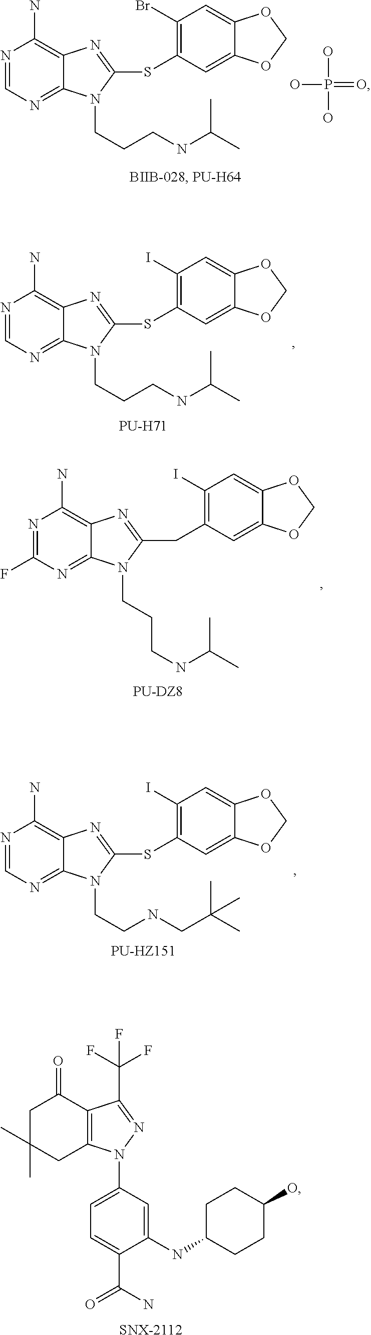

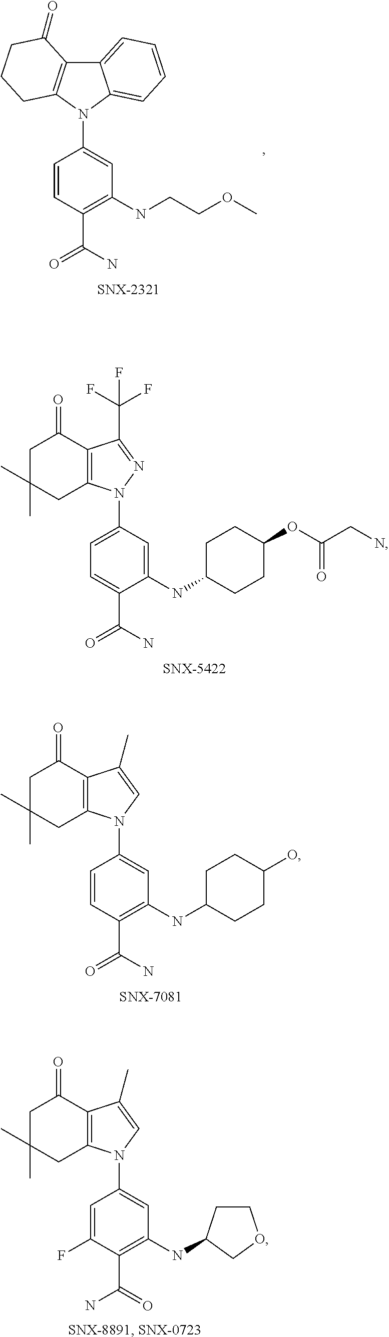

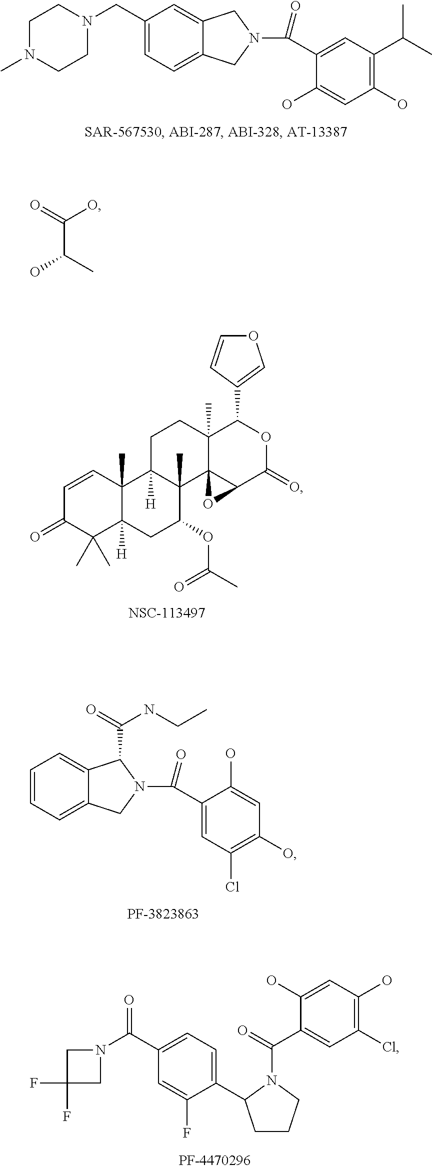

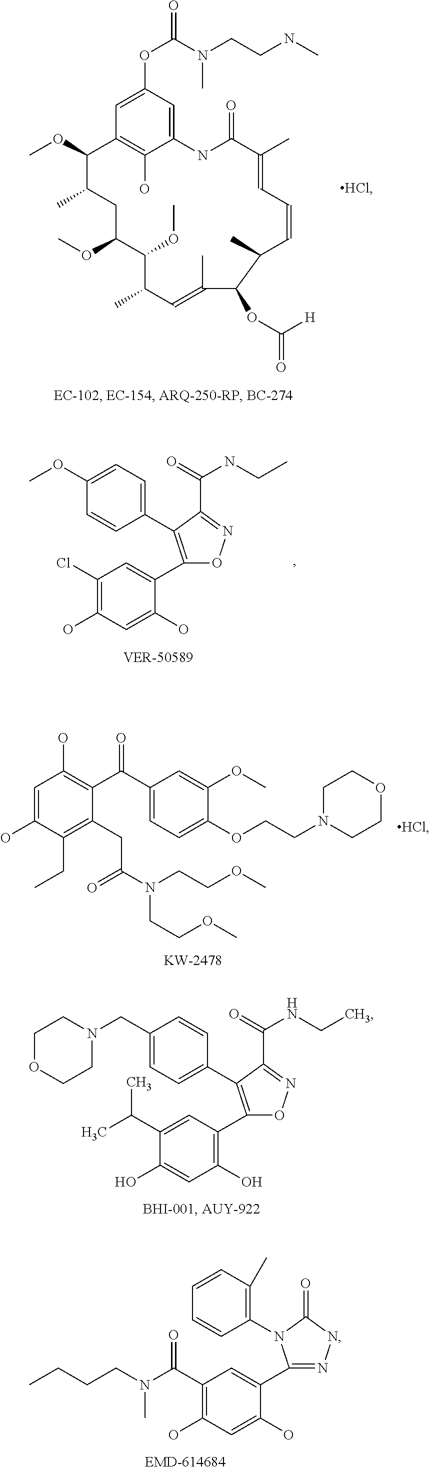

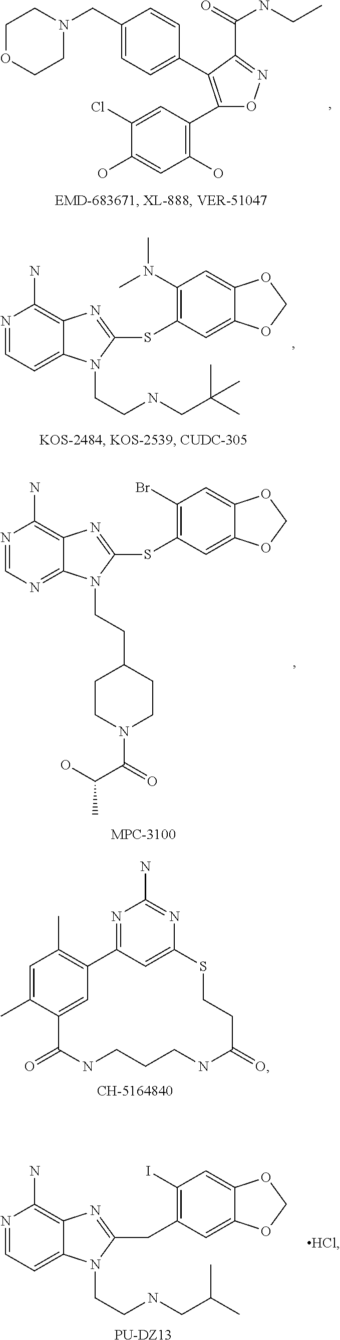

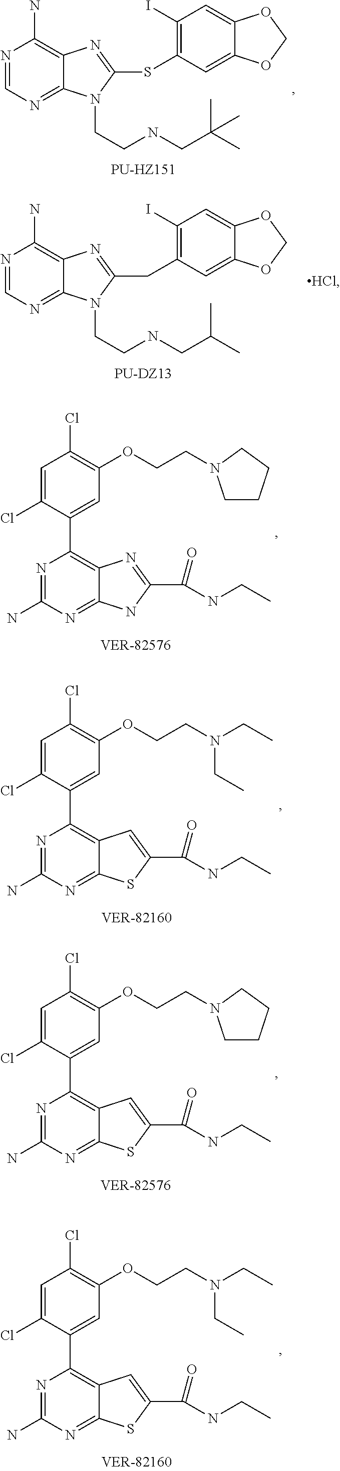

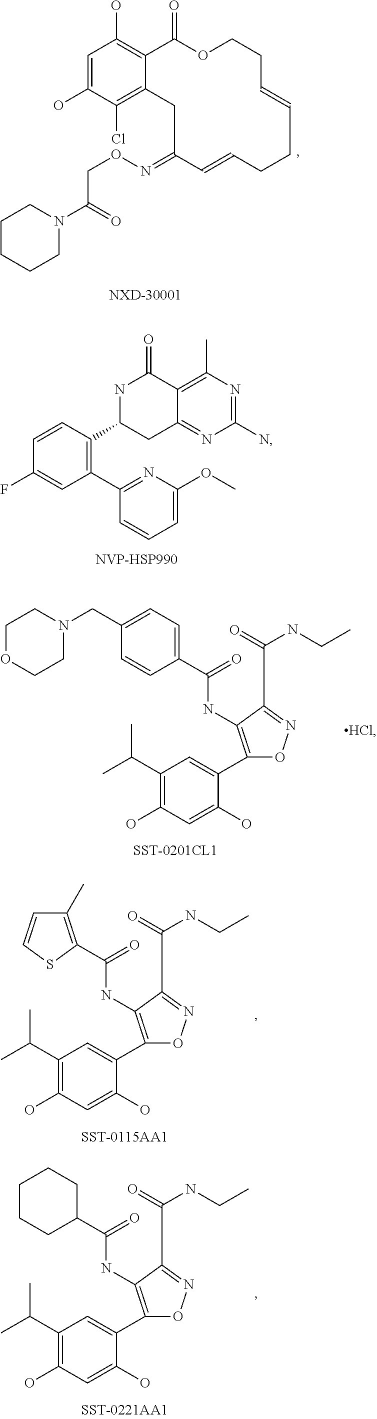

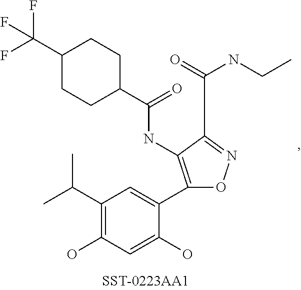 [SMILES: CCNC(=O)c1noc(-c2cc(C(C)C)c(O)cc2O)c1NC(=O)C1CCC(C(F)(F)F)CC1]

novobiocin (a C-terminal Hsp90i.), or a tautomer/derivative/analog thereof. The selection of other Hsp90-targeting moieties will be within the grasp of one of ordinary skill in the art. Likewise, the selection of binding moieties suitable for other molecular targets and/or other applications will be within the ability of one of ordinary skill in the art.

Additionally Hsp90 targeting moieties can be used to construct SDC-TRAP molecules for the treatment of inflammation. For example, binding moieties comprising the compounds shown in Tables 5, 6, and 7 of U.S. Patent Publication 2010/0280032, which is incorporated herein by reference in its entirety, or compounds of any formula therein, or tautomers, pharmaceutically acceptable salts, solvates, clathrates, hydrates, polymorphs or prodrugs thereof, inhibit the activity of Hsp90 and, thereby cause the degradation of Hsp90 client proteins. Any of these compounds may be coupled to an effector molecule to form an SDC-TRAP. The glucocorticoid receptor is a client protein of Hsp90 and binds to Hsp90 when it is in the conformation that is able to bind glucocorticoid ligands such as cortisol. Once a glucocorticoid binds to GR, the receptor disassociates with Hsp90 and translocates to the nucleus where it modulates gene expression to reduce inflammatory responses such as proinflammatory cytokine production. Thus, glucocorticoids may be given to patients in need of immunosuppression and patients with inflammatory and autoimmune disorders. Unfortunately, although glucocorticoids are effective at relieving inflammation, they have a number of severe side effects including osteoporosis, muscle wasting, hypertension, insulin resistance, truncal obesity and fat redistribution, and inhibition of wound repair. Inhibition of Hsp90 causes changes in GR activity which results in reduction of inflammatory responses similar to those seen for glucocorticoids. However, since the mechanism for reducing inflammation is different than that of glucocorticoids, it is expected that some or all of the side effects of glucocorticoid treatment will be reduced or eliminated.

### Effector Moieties

An effector moiety can be any therapeutic or imaging agent that can be conjugated to a binding moiety and, in a thus conjugated state, delivered to a molecular target of the binding moiety. An effector molecule can, in some cases, require a linking moiety for conjugation (e.g., cannot be directly conjugated to a binding moiety). Similarly, an effector molecule can, in some cases, impede or reduce the ability of the binding moiety and/or SDC-TRAP to reach a target as long as the SDC-TRAP can still effect the target. However, in preferred embodiments, an effector moiety is readily conjugatable and may benefits delivery to, and effecting, of the target.

In various embodiments, an SDC-TRAP, via an effector moiety, can have other ways of cell penetration than simple passive diffusion. Such an example is an SDC-TRAP including an antifolate or fragments thereof (e.g., temozolamide, mitozolamide, nitrogen mustards, estramustine, or chloromethine) as the effector moiety. In this case, a conjugate of a binding moiety (e.g., Hsp90 inhibitor) with pemetrexed (or its folate-recognizing fragment) can undergo folate receptor mediated endocytosis rather than passive diffusion. Once in a target cell, the SDC-TRAP can bind the molecular target (e.g., Hsp90 protein) via its binding moiety (e.g., Hsp90 inhibitor).

As described in greater detail below, an effector moiety can comprise a region that can be modified and/or participate in covalent linkage to a binding moiety without substantially adversely affecting the binding moiety's ability to bind to its target. An effector moiety can be a pharmaceutical molecule or a derivative thereof, which essentially retains activity while conjugated to a binding moiety. It will be appreciated that drugs with otherwise good and desirable activity can prove challenging to administer conventionally (e.g., due to poor bioavailability or undesirable side-effects in vivo prior to reaching their target)—such drugs can be “reclaimed” for use as effector moieties in the SDC-TRAPs of the present invention.

Examples of effector moieties include: peptidyl-prolyl isomerase ligands, e.g., FK506; rapamycin, cyclosporin A and the like; steroid hormone receptor ligands, e.g., naturally occurring steroid hormones, such as estrogen, progestin, testosterone, and the like, as well as synthetic derivatives and mimetics thereof; binding moieties that bind to cytoskeletal proteins, e.g., antimitotic agents, such as taxanes, colchicine, colcemid, nocadozole, vinblastine, and vincristine, actin binding agents, such as cytochalasin, latrunculin, phalloidin, and the like; lenalidomide, pomalidomide, camptothecins including

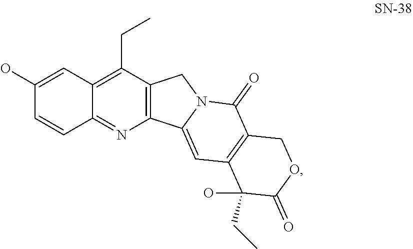 [SMILES: CCc1c2c(nc3ccc(O)cc13)-c1cc3c(c(=O)n1C2)COC(=O)[C@]3(O)CC]

topotecan, combretastatins, capecitabine, gemcitabine, vinca alkaloids, platinum-containing compounds, metformin, HDAC inhibitors (e.g., suberoylanilidehydroxamic acid (SAHA)), thymidylate synthase inhibitors such as methotrexate, pemetrexed, and raltitrexed; nitrogen mustards such as bendamustine and melphalan; 5-fluorouracil (5-FU) and its derivatives; and agents used in ADC drugs, such as vedotin and DM1, or a tautomer/derivative/analog thereof.

The effector moiety may be obtained from a library of naturally occurring or synthetic molecules, including a library of compounds produced through combinatorial means, i.e., a compound diversity combinatorial library. When obtained from such libraries, the effector moiety employed will have demonstrated some desirable activity in an appropriate screening assay for the activity. It is contemplated that in other embodiments, the pharmaceutical conjugate may include more than one effector moiety(ies), providing the medicinal chemist with more flexibility. The number of effector moieties linked to the binding moiety (e.g., Hsp90-targeting moiety) will generally only be limited by the number of sites on the binding moiety (e.g., Hsp90-targeting moiety) and/or any linking moiety available for linking to an effector moiety; the steric considerations, e.g., the number of effector moieties than can actually be linked to the binding moiety (e.g., Hsp90-targeting moiety); and that the ability of the pharmaceutical conjugate to bind to the molecular target (e.g., Hsp90 protein) is preserved.

Specific drugs from which the effector moiety may be derived include: psychopharmacological agents, such as central nervous system depressants, e.g., general anesthetics (barbiturates, benzodiazepines, steroids, cyclohexanone derivatives, and miscellaneous agents), sedative-hypnotics (benzodiazepines, barbiturates, piperidinediones and triones, quinazoline derivatives, carbamates, aldehydes and derivatives, amides, acyclic ureides, benzazepines and related drugs, phenothiazines, etc.), central voluntary muscle tone modifying drugs (anticonvulsants, such as hydantoins, barbiturates, oxazolidinediones, succinimides, acylureides, glutarimides, benzodiazepines, secondary and tertiary alcohols, dibenzazepine derivatives, valproic acid and derivatives, GABA analogs, etc.), analgesics (morphine and derivatives, oripavine derivatives, morphinan derivatives, phenylpiperidines, 2,6-methane-3-benzazocaine derivatives, diphenylpropylamines and isosteres, salicylates, p-aminophenol derivatives, 5-pyrazolone derivatives, arylacetic acid derivatives, fenamates and isosteres, etc.) and antiemetics (anticholinergics, antihistamines, antidopaminergics, etc.); central nervous system stimulants, e.g., analeptics (respiratory stimulants, convulsant stimulants, psychomotor stimulants), narcotic antagonists (morphine derivatives, oripavine derivatives, 2,6-methane-3-benzoxacine derivatives, morphinan derivatives) nootropics; psychopharmacological/psychotropics, e.g., anxiolytic sedatives (benzodiazepines, propanediol carbamates) antipsychotics (phenothiazine derivatives, thioxanthine derivatives, other tricyclic compounds, butyrophenone derivatives and isosteres, diphenylbutylamine derivatives, substituted benzamides, arylpiperazine derivatives, indole derivatives, etc.), antidepressants (tricyclic compounds, MAO inhibitors, etc.); respiratory tract drugs, e.g., central antitussives (opium alkaloids and their derivatives); immunosuppressive agents; pharmacodynamic agents, such as peripheral nervous system drugs, e.g., local anesthetics (ester derivatives, amide derivatives); drugs acting at synaptic or neuroeffector junctional sites, e.g., cholinergic agents, cholinergic blocking agents, neuromuscular blocking agents, adrenergic agents, antiadrenergic agents; smooth muscle active drugs, e.g., spasmolytics (anticholinergics, musculotropic spasmolytics), vasodilators, smooth muscle stimulants; histamines and antihistamines, e.g., histamine and derivative thereof (betazole), antihistamines (H₁-antagonists, H₂-antagonists), histamine metabolism drugs; cardiovascular drugs, e.g., cardiotonics (plant extracts, butenolides, pentadienolids, alkaloids from erythrophleum species, ionophores, -adrenoceptor stimulants, etc.), antiarrhythmic drugs, antihypertensive agents, antilipidemic agents (clofibric acid derivatives, nicotinic acid derivatives, hormones and analogs, antibiotics, salicylic acid and derivatives), antivaricose drugs, hemostyptics; chemotherapeutic agents, such as anti-infective agents, e.g., ectoparasiticides (chlorinated hydrocarbons, pyrethins, sulfurated compounds), anthelmintics, antiprotozoal agents, antimalarial agents, antiamebic agents, antileiscmanial drugs, antitrichomonal agents, antitrypanosomal agents, sulfonamides, antimycobacterial drugs, antiviral chemotherapeutics, etc., and cytostatics, i.e., antineoplastic agents or cytotoxic drugs, such as alkylating agents, e.g., Mechlorethamine hydrochloride (Nitrogen Mustard, Mustargen, HN2), Cyclophosphamide (Cytovan, Endoxana), Ifosfamide (IFEX), Chlorambucil (Leukeran), Melphalan (Phenylalanine Mustard, L-sarcolysin, Alkeran, L-PAM), Busulfan (Myleran), Thiotepa (Triethylenethiophosphoramide), Carmustine (BiCNU, BCNU), Lomustine (CeeNU, CCNU), Streptozocin (Zanosar) and the like; plant alkaloids, e.g., Vincristine (Oncovin), Vinblastine (Velban, Velbe), Paclitaxel (Taxol), and the like; antimetabolites, e.g., Methotrexate (MTX), Mercaptopurine (Purinethol, 6-MP), Thioguanine (6-TG), Fluorouracil (5-FU), Cytarabine (Cytosar-U, Ara-C), Azacitidine (Mylosar, 5-AZA) and the like; antibiotics, e.g., Dactinomycin (Actinomycin D, Cosmegen), Doxorubicin (Adriamycin), Daunorubicin (duanomycin, Cerubidine), Idarubicin (Idamycin), Bleomycin (Blenoxane), Picamycin (Mithramycin, Mithracin), Mitomycin (Mutamycin) and the like, and other anticellular proliferative agents, e.g., Hydroxyurea (Hydrea), Procarbazine (Mutalane), Dacarbazine (DTIC-Dome), Cisplatin (Platinol) Carboplatin (Paraplatin), Asparaginase (Elspar) Etoposide (VePesid, VP-16-213), Amsarcrine (AMSA, m-AMSA), Mitotane (Lysodren), Mitoxantrone (Novatrone), and the like; anti-inflammatory agents; antibiotics, such as: aminoglycosides, e.g., amikacin, apramycin, arbekacin, bambermycins, butirosin, dibekacin, dihydrostreptomycin, fortimicin, gentamicin, isepamicin, kanamycin, micronomcin, neomycin, netilmicin, paromycin, ribostamycin, sisomicin, spectinomycin, streptomycin, tobramycin, trospectomycin; amphenicols, e.g., azidamfenicol, chloramphenicol, florfenicol, and theimaphenicol; ansamycins, e.g., rifamide, rifampin, rifamycin, rifapentine, rifaximin; β-lactams, e.g., carbacephems, carbapenems, cephalosporins, cehpamycins, monobactams, oxaphems, penicillins; lincosamides, e.g., clinamycin, lincomycin; macrolides, e.g., clarithromycin, dirthromycin, erythromycin, etc.; polypeptides, e.g., amphomycin, bacitracin, capreomycin, etc.; tetracyclines, e.g., apicycline, chlortetracycline, clomocycline, etc.; synthetic antibacterial agents, such as 2,4-diaminopyrimidines, nitrofurans, quinolones and analogs thereof, sulfonamides, sulfones; antifungal agents, such as: polyenes, e.g., amphotericin B, candicidin, dermostatin, filipin, fungichromin, hachimycin, hamycin, lucensomycin, mepartricin, natamycin, nystatin, pecilocin, perimycin; synthetic antifungals, such as allylamines, e.g., butenafine, naftifine, terbinafine; imidazoles, e.g., bifonazole, butoconazole, chlordantoin, chlormidazole, etc., thiocarbamates, e.g., tolciclate, triazoles, e.g., fluconazole, itraconazole, terconazole; anthelmintics, such as: arecoline, aspidin, aspidinol, dichlorophene, embelin, kosin, napthalene, niclosamide, pelletierine, quinacrine, alantolactone, amocarzine, amoscanate, ascaridole, bephenium, bitoscanate, carbon tetrachloride, carvacrol, cyclobendazole, diethylcarbamazine, etc.; antimalarials, such as: acedapsone, amodiaquin, arteether, artemether, artemisinin, artesunate, atovaquone, bebeerine, berberine, chirata, chlorguanide, chloroquine, chlorprogaunil, cinchona, cinchonidine, cinchonine, cycloguanil, gentiopicrin, halofantrine, hydroxychloroquine, mefloquine hydrochloride, 3-methylarsacetin, pamaquine, plasmocid, primaquine, pyrimethamine, quinacrine, quinidine, quinine, quinocide, quinoline, dibasic sodium arsenate; and antiprotozoan agents, such as: acranil, tinidazole, ipronidazole, ethylstibamine, pentamidine, acetarsone, aminitrozole, anisomycin, nifuratel, tinidazole, benzidazole, suramin, and the like.

### Conjugation and Linking Moieties

Binding moieties and effector moieties of the present invention can be conjugated, for example, through a linker or linking moiety L, where L may be either a bond or a linking group. For example, in various embodiments, a binding moiety and an effector moiety are bound directly or are parts of a single molecule. Alternatively, a linking moiety can provide a covalent attachment between a binding moiety and effector moiety. A linking moiety, as with a direct bond, can achieve a desired structural relationship between a binding moiety and effector moiety and or an SDC-TRAP and its molecular target. A linking moiety can be inert, for example, with respect to the targeting of a binding moiety and biological activity of an effector moiety.

Appropriate linking moieties can be identified using the affinity, specificity, and/or selectivity assays described herein. Linking moieties can be selected based on size, for example, to provide an SDC-TRAP with size characteristics as described above. In various embodiments, a linking moiety can be selected, or derived from, known chemical linkers. Linking moieties can comprise a spacer group terminated at either end with a reactive functionality capable of covalently bonding to the drug or ligand moieties. Spacer groups of interest include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linking moieties that may find use in the SDC-TRAPs include disulfides and stable thioether moieties.

In various embodiments, a linking moiety is cleavable, for example enzymatically cleavable. A cleavable linker can be used to release an effector moiety inside a target cell after the SDC-TRAP is internalized. The susceptibility of a linking moiety to cleavage can be used to control delivery of an effector molecule. For example, a linking moiety can be selected to provide extended or prolonged release of an effector moiety in a target cell over time (e.g., a carbamate linking moiety may be subject to enzymatic cleavage by a carboxylesterase via the same cellular process used to cleave other carbamate prodrugs like capecitabine or irinotecan). In these, and various other embodiments, a linking moiety can exhibit sufficient stability to ensure good target specificity and low systemic toxicity, but not so much stability that it results in lowering the potency and efficacy of the SDC-TRAP.

Exemplary linkers are described in U.S. Pat. No. 6,214,345 (Bristol-Myers Squibb), U.S. Pat. Appl. 2003/0096743 and U.S. Pat. Appl. 2003/0130189 (both to Seattle Genetics), de Groot et al., J. Med. Chem. 42, 5277 (1999); de Groot et al. J. Org. Chem. 43, 3093 (2000); de Groot et al., J. Med. Chem. 66, 8815, (2001); WO 02/083180 (Syntarga); Carl et al., J. Med. Chem. Lett. 24, 479, (1981); Dubowchik et al., Bioorg & Med. Chem. Lett. 8, 3347 (1998) and Doronina et al. BioConjug Chem. 2006; Doronina et al. Nat Biotech 2003.

In one embodiment, the SDC-TRAP comprises ganetespib or its tautomer as a binding moiety, and SN-38 or its fragment/derivative/analog as an effector moiety. One non-limiting example is SDC-TRAP-0063. The term SDC-TRAP-0063 includes a compound having a structure of:

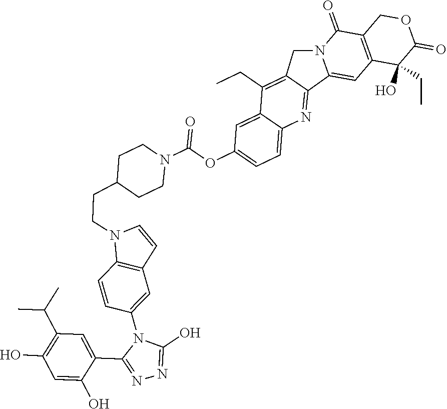 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]

or its tautomer:

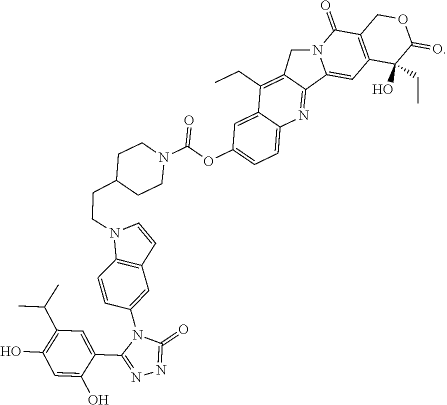

### Methods of Making Pharmaceutical Conjugates

The pharmaceutical conjugates, i.e., SDC-TRAPs, of the invention may be prepared using any convenient methodology. In a rational approach, the pharmaceutical conjugates are constructed from their individual components, binding moiety, in some cases a linker, and effector moiety. The components can be covalently bonded to one another through functional groups, as is known in the art, where such functional groups may be present on the components or introduced onto the components using one or more steps, e.g., oxidation reactions, reduction reactions, cleavage reactions and the like. Functional groups that may be used in covalently bonding the components together to produce the pharmaceutical conjugate include: hydroxy, sulfhydryl, amino, and the like. The particular portion of the different components that are modified to provide for covalent linkage will be chosen so as not to substantially adversely interfere with that components desired binding activity, e.g., for the effector moiety, a region that does not affect the target binding activity will be modified, such that a sufficient amount of the desired drug activity is preserved. Where necessary and/or desired, certain moieties on the components may be protected using blocking groups, as is known in the art, see, e.g., Green & Wuts, Protective Groups in Organic Synthesis (John Wiley & Sons) (1991).

Alternatively, the pharmaceutical conjugate can be produced using known combinatorial methods to produce large libraries of potential pharmaceutical conjugates which may then be screened for identification of a bifunctional, molecule with the pharmacokinetic profile. Alternatively, the pharmaceutical conjugate may be produced using medicinal chemistry and known structure-activity relationships for the targeting moiety and the drug. In particular, this approach will provide insight as to where to join the two moieties to the linker.

A number of exemplary methods for preparing SDC-TRAP molecules are set forth in the examples. As one of skill in the art will understand, the exemplary methods set forth in the examples can be modified to make other SDC-TRAP molecules.

### Methods of Use, Pharmaceutical Preparations, and Kits

The pharmaceutical conjugates find use in treatment of a host condition, e.g., a disease condition. In these methods, an effective amount of the pharmaceutical conjugate is administered to the host, where “effective amount” means a dosage sufficient to produce the desired result, e.g., an improvement in a disease condition or the symptoms associated therewith. In many embodiments, the amount of drug in the form of the pharmaceutical conjugate that need be administered to the host in order to be an effective amount will vary from that which must be administered in free drug form. The difference in amounts may vary, and in many embodiments may range from two-fold to ten-fold. In certain embodiments, e.g., where the resultant modulated pharmacokinetic property or properties result(s) in enhanced activity as compared to the free drug control, the amount of drug that is an effective amount is less than the amount of corresponding free drug that needs to be administered, where the amount may be two-fold, usually about four-fold and more usually about ten-fold less than the amount of free drug that is administered.

The pharmaceutical conjugate may be administered to the host using any convenient means capable of producing the desired result. Thus, the pharmaceutical conjugate can be incorporated into a variety of formulations for therapeutic administration. More particularly, the pharmaceutical conjugate of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the pharmaceutical conjugate can be achieved in various ways, including oral, buccal, rectal, parenteral, intraperitoneal, intradermal, transdermal, intracheal, etc., administration. In pharmaceutical dosage forms, the pharmaceutical conjugate may be administered alone or in combination with other pharmaceutically active compounds.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The conjugates or particles of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfate, sodium metabisulfite, potassium sulfite, potassium metabisulfite, GLYDANT PLUS®, PHENONIP®, methylparaben, GERMALL®115, GERMABEN®II, NEOLONE™ KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

The subject methods find use in the treatment of a variety of different disease conditions. In certain embodiments, of particular interest is the use of the subject methods in disease conditions where an active agent or drug having desired activity has been previously identified, but which active agent or drug does not bind to its target with desired affinity and/or specificity. With such active agents or drugs, the subject methods can be used to enhance the binding affinity and/or specificity of the agent for its target.

The specific disease conditions treatable by with the subject bifunctional compounds are as varied as the types of drug moieties that can be present in the pharmaceutical conjugate. Thus, disease conditions include cellular proliferative diseases, such as neoplastic diseases, autoimmune diseases, central nervous system or neurodegenerative diseases, cardiovascular diseases, hormonal abnormality diseases, infectious diseases, and the like.

By treatment is meant at least an amelioration of the symptoms associated with the disease condition afflicting the host, where amelioration is used in a broad sense to refer to at least a reduction in the magnitude of a parameter, e.g., symptom, associated with the pathological condition being treated, such as inflammation and pain associated therewith. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

Methods of use of the invention extend beyond strict treatment of a disease. For example, the invention includes uses in a clinical or research setting to diagnose a subject, select a subject for therapy, select a subject for participation in a clinical trial, monitor the progression of a disease, monitor the effect of therapy, to determine if a subject should discontinue or continue therapy, to determine if a subject has reached a clinical end point, and to determine recurrence of a disease. The invention also includes uses in conducting research to identify effective interacting moieties and/or effector moieties and/or combinations thereof, to identify effective dosing and dose scheduling, to identify effective routes of administration, and to identify suitable targets (e.g., diseases susceptible to particular treatment).

A variety of hosts are treatable according to the subject methods. Generally such hosts are “mammals” or “mammalian,” where these terms are used broadly to describe organisms which are within the class Mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The invention provides kits for treating a subject in need thereof comprising at least one SDC-TRAP and instruction for administering a therapeutically effective amount of the at least one SDC-TRAP to the subject, thereby treating the subject. The invention also provides kits for imaging, diagnosing, and/or selecting a subject comprising at least one SDC-TRAP and instruction for administering an effective amount of at least one SDC-TRAP to the subject, thereby imaging, diagnosing, and/or selecting the subject.

Kits with unit doses of the pharmaceutical conjugate, usually in oral or injectable doses and often in a storage stable formulation, are provided. In such kits, in addition to the containers containing the unit doses, an informational package insert describing the use and attendant benefits of the drugs in treating pathological condition of interest will be included. Preferred compounds and unit doses are those described herein above.

The invention also provides methods for treatment of a disease or disorder in which the subject to be treated is selected for treatment based on the presence of, or the overexpression of, a particular protein. For example, subjects may be selected for treatment of cancer based on the presence of greater the normal levels of Hsp90. In this case, subjects would be administered an SDC-TRAP that comprises a binding moiety that selectively binds to Hsp90.

The invention provides methods of treating or preventing an inflammatory disorder in a subject, comprising administering to the subject an effective amount of a compound represented by any one of formula (I) through (LXXII), or any embodiment thereof, or a compound shown in Table 5, 6, or 7 as disclosed in U.S. Patent Publication 2010/0280032. In one embodiment, the compound or binding moiety or SDC-TRAP may be administered to a human to treat or prevent an inflammatory disorder. In another embodiment, the inflammatory disorder is selected from the group consisting of transplant rejection, skin graft rejection, arthritis, rheumatoid arthritis, osteoarthritis and bone diseases associated with increased bone resorption; inflammatory bowel disease, ileitis, ulcerative colitis, Barrett's syndrome, Crohn's disease; asthma, adult respiratory distress syndrome, chronic obstructive airway disease; corneal dystrophy, trachoma, onchocerciasis, uveitis, sympathetic ophthalmitis, endophthalmitis; gingivitis, periodontitis; tuberculosis; leprosy; uremic complications, glomerulonephritis, nephrosis; sclerodermatitis, psoriasis, eczema; chronic demyelinating diseases of the nervous system, multiple sclerosis, AIDS-related neurodegeneration, Alzheimer's disease, infectious meningitis, encephalomyelitis, Parkinson's disease, Huntington's disease, amyotrophic lateral sclerosis viral or autoimmune encephalitis; autoimmune disorders, immune-complex vasculitis, systemic lupus and erythematodes; systemic lupus erythematosus (SLE); cardiomyopathy, ischemic heart disease hypercholesterolemia, atherosclerosis, preeclampsia; chronic liver failure, brain and spinal cord trauma. In another embodiment, an SDC-TRAP, or a compound shown in Table 5, 6, or 7 as disclosed in U.S. Patent Publication 2010/0280032, is administered with an additional therapeutic agent. In another embodiment, the additional therapeutic agent may an anti-inflammatory agent.

In one embodiment, an SDC-TRAP that is administered to a subject but does not enter a target cell is rapidly cleared from the body. In this embodiment, the SDC-TRAP that does not enter a target cell is rapidly cleared in order to reduce the toxicity due to the components of the SDC-TRAP, the degradation products of the SDC-TRAP or the SDC-TRAP molecule. Clearance rate can be determined by measuring the plasma concentration of the SDC-TRAP molecule as a function of time.

Likewise, SDC-TRAP molecules that enter non-targeted cells by passive diffusion rapidly exit the non-targeted cell or tissue and are either eliminated from the subject or proceed to enter and be retained a targeted cell or tissue. For example, an SDC-TRAP that is intended to treat tumor cells and is targeted to tumor cells that overexpress, for example, Hsp90 will accumulate selectively in tumor cells that overexpress Hsp90. Accordingly, very low levels of this exemplary SDC-TRAP will be present in non-tumor tissue such as normal lung tissue, heart, kidney, and the like. In one embodiment, the safety of the SDC-TRAP molecules of the invention can be determined by their lack of accumulation in non-targeted tissue. Conversely, the safety of the SDC-TRAP molecules of the invention can be determined by their selective accumulation in the targeted cells and/or tissue.

In one example, a pharmaceutical composition comprising an effective amount of SDC-TRAP-0063 Sodium, a tautomer thereof, or a pharmaceutically acceptable salt thereof, and 5% Mannitol is provided. The pharmaceutical composition has a pH in the range of about 9.4 to about 10.3. The concentration of SDC-TRAP-0063 Sodium, a tautomer thereof, or a pharmaceutically acceptable salt thereof is in the range of around 1 mg/mL to around 20 mg/mL, such as about 3 mg/mL, 6 mg/mL, or 12 mg/mL.

### EXAMPLES

The following examples, which are briefly summarized and then discussed in turn below, are offered by way of illustration and not by way of limitation.

### Example 1: Synthesis of SDC-TRAP-0063

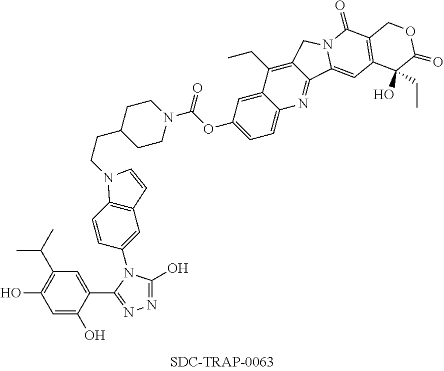 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]

((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate) or its tautomer.

A synthesis scheme of the synthesis of SDC-TRAP-0063 is provided in Example 6 of PCT Application No. PCT/US2013/036783. The person of ordinary skill in the art would be able, without undue experimentation, to adapt this synthetic scheme for making other targeting molecule conjugates within the scope of the invention.

### Example 2: Salt Form and Formulation of HSP90 Binding Drug Conjugate

In solution, SDC-TRAP-0063 contains a lactone ring at pH-dependent equilibrium with the corresponding open chain carboxylic acid form. At high pH (above pH of 9.3, pKa value) the equilibrium shifts toward an open ring carboxylic acid form and at low pH it shifts toward the closed ring lactone form shown below:

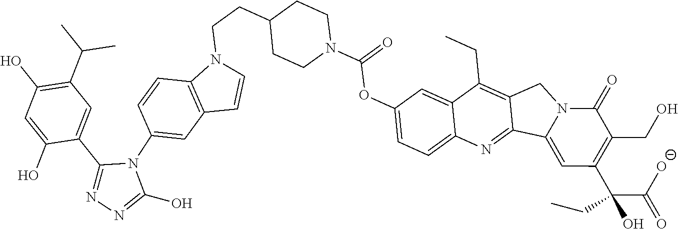 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc([C@@](O)(CC)C(=O)[O-])c(CO)c(=O)n1C2]

The open ring carboxylic acid form may form a salt with cationic ions include, but not limited to, lithium, aluminum, calcium, magnesium, potassium, sodium, zinc, barium, bismuth, benethamine, diethylamine, tromethamine, benzathid, chloroprocaine, choline, diethanolamine, ethylenediamine, meglumine, or procaine.

### Sodium Salt Derivative of SDC-TRAP-0063

The sodium salt (SDC-TRAP-0063 Sodium or SDC-TRAP-0063 Na) of the carboxylic acid derivative has a structure of

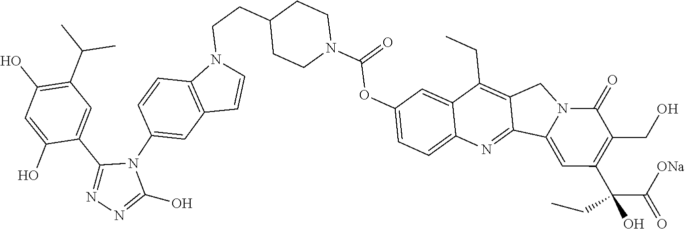

(Sodium (S)-2-(2-((4-(2-(5-(3-(2,4-dihydroxy-5-isopropylphenyl)-5-hydroxy-4H-1,2,4-triazol-4-yl)-1H-indol-1-yl)ethyl)piperidine-1-carbonyl)oxy)-12-ethyl-8-(hydroxymethyl)-9-oxo-9,11-dihydroindolizino[1,2-b]quinolin-7-yl)-2-hydroxybutanoate) or its tautomer:

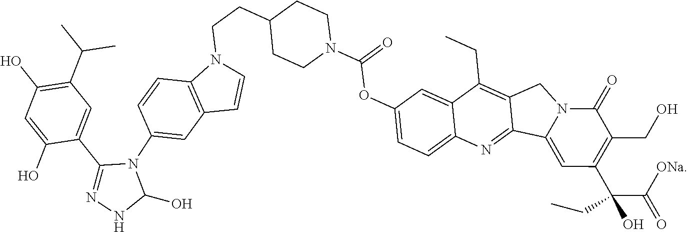

Structure of SDC-TRAP-0063 in both lactone and sodium salt form:

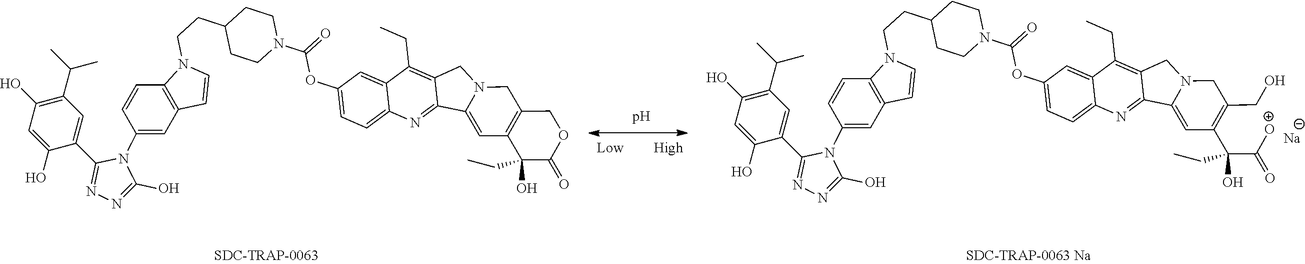

SDC-TRAP-0063 drug substance is isolated and stored in the lactone form and SDC-TRAP-0063 Sodium drug product is converted and stored in the carboxylic acid sodium salt form.

SDC-TRAP-0063 can be prepared with the following process: a portion of tert-butanol was melted at 28-32° C. and dispensed into an 8-liter glass mixing vessel jacketed at 28-32° C. SDC-TRAP-0063 powder was added slowly into the stirring tert-butanol and mixed for at least 20 minutes. The quantity of SDC-TRAP-0063 added was determined gravimetrically and the target drug product batch size was calculated. A second portion of tert-butanol was then added by weight quantity sufficient (Q.S. or QS) and mixed for at least 15 minutes with a ˜6″ magnetic stir bar to adequately wet and suspend it. 0.3 normal aqueous sodium hydroxide solution was then slowly added and allowed to mix for at least an additional 1 hour. Complete dissolution of SDC-TRAP-0063 powder was confirmed by visual observation both while mixing and while the mixer was stopped. Water for Injection (WFI) was then dispensed up to ˜95% of the target total batch volume and mixed for 20 minutes. A sample was taken and measured to ensure pH was greater than or equal to 9.8, with option to adjust by incremental addition of 5-gram aliquots of 0.3 normal aqueous sodium hydroxide solution with at least 15 minutes of mixing if necessary. Water for Injection was again added to weight QS and mixed for 15 minutes to complete compounding of the bulk drug solution. The 8 liter glass mixing vessel jacket temperature was then reduced to within 20-25° C. Product sterilization was achieved by filtration through one at least two Millipore Opticap XL3 0.2 μm filters in series, and samples were taken immediately pre-filter for microbial enumeration testing. Depyrogenated 10 milliliter nominal size borosilicate glass vials were then aseptically filled with 1.1 milliliters of bulk drug solution per vial. Vials were stoppered into the lyophilization position and loaded into a lyophilizer. Vials were lyophilized per the recipe in Table 1, and fully stoppered. Vials were aseptically removed from the lyophilizer and caps were crimped to seal the vials. Exterior vial washing and visual inspection were conducted to complete production of the drug product in its primary enclosure.

TABLE 1
Lyophilization steps and conditions

| Step # | Step Description | Temperature | Pressure | Duration |
|---|---|---|---|---|
| 1 | Loading | 5° C. | Atmospheric | Not applicable |
| 2 | Freezing | 5° C. | Atmospheric | 120 minutes |
| 3 | Freezing ramp | 5° C. to −50° C. | Atmospheric | 120 minutes |
| 4 | Freezing | −50° C. | Atmospheric | 210 minutes |
| 5 | Freezing ramp | −50° C. to −40° C. | Atmospheric | 15 minutes |
| 6 | Evacuation | −40° C. | 80 μbar | Not applicable |
| 7 | Primary drying ramp | −40° C. to −15° C. | 80 μbar | 50 minutes |
| 8 | Primary drying | −15° C. | 80 μbar | 2,520 minutes |
| 9 | Secondary drying ramp | −15° C. to 25° C. | 80 μbar | 156 minutes |
| 10 | Secondary drying | 25° C. | 80 μbar | 960 minutes |
| 11 | Pre-aeration with nitrogen | 25° C. | 800 μbar | Not applicable |
| 12 | Stoppering | 25° C. | 800 μbar | Not applicable |
| 13 | Aeration with nitrogen | 25° C. | Atmospheric | Not applicable |
| 14 | Unloading* | 25° C. | Atmospheric | Not applicable |

* If unloading is not immediate, maintain the shelves at 5° C.; before starting unloading, move the shelf temperature to the unloading temperature.

During the manufacturing process, SDC-TRAP-0063 was converted to SDC-TRAP-0063 Sodium, which is the dominant form at pH above 9.3. SDC-TRAP-0063 Sodium drug product was aseptically manufactured as a sterile-filtered solution that was lyophilized. The composition of the lyophilized drug product is shown below:

| Ingredient | Role | Amount (mg/vial) |
|---|---|---|
| SDC-TRAP-0063 sodium | Active | 105 |

This solution is filled to deliver 105 mg/vial into a container closure system consisting of a USP Type 1 clear glass vial, stopper, and overseal. The drug product is stored at 2° C. to 8° C., away from light. Prior to administration the lyophilized powder is reconstituted with Water for Injection and then is further diluted in 5% Mannitol, USP to the target concentration prior to use. SDC-TRAP-0063 Sodium may have a concentration of between about 20 to about 25 mg/mL, about 25 to about 50 mg/mL, between about 50 to about 100 mg/mL, between about 100 to about 150 mg/mL, or between about 150 to 200 mg/mL. The drug product is intended for intravenous administration by infusion.

The reconstituted solution of SDC-TRAP-0063 Sodium has a pH of about 10.0. This solution is diluted to the target dose in 5% Mannitol, USP. The pH of the infusion solution depends on the concentration of SDC-TRAP-0063 Sodium in the diluted infusion solution. Across the dose ranges employed in the clinical study protocol, the volume of the diluted infusion solution administered will range from 50 to 500 mL, and the pH will range from 8.1 to 9.6. In order to reduce the potential risks of injection site pain and/or damage to the venous endothelium during IV administration, a central venous access line is used for administration of the diluted SDC-TRAP-0063 Sodium.

### Example 3: Modified Dosing Solution and Administration Process

A new, more robust and patient-friendly formulation of dosing solution was developed. During this development, it was established that the pH of the dosing solution is driven by the concentration of SDC-TRAP-0063 Sodium and increasing the SDC-TRAP-0063 Sodium concentration allows better control of the solubility and pH of dosing solutions.
increased pH (>9.4)

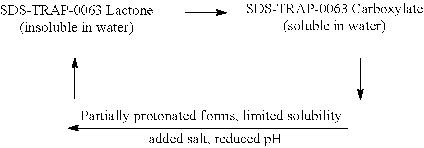

It was found that the pH control and solubility of SDC-TRAP-0063 Sodium appears to mitigate the risk of precipitation in 5% Mannitol, compared to 0.9% Sodium chloride. It is believed that a common ion effect is decreasing the solubility of SDC-TRAP-0063 Sodium in 0.9% Sodium Chloride solution. The change of pH from 8.6-8.7 observed for dosing solutions in 0.9% Sodium chloride to the pH range of 9.4-10.2 observed for SDC-TRAP-0063 Sodium dosing solutions in 5% Mannitol provides sufficient solubility and stability for clinical use.

In the study described below, it was found that using 5% Mannitol as the diluent and at higher concentrations of SDC-TRAP-0063 Sodium than used in 0.9% Sodium Chloride provides a stable solution suitable for clinical dosing. The roles of solution pH and Mannitol in preventing precipitation have been evaluated through multiple experiments. The data justify the use of Mannitol as diluent and the increased concentration of SDC-TRAP-0063 Sodium to enhance drug solubility.

### Study Design and Results

As discussed above, the solubility of SDC-TRAP-0063 Sodium is driven primarily by pH. SDC-TRAP-0063 Sodium has very high solubility in water of >52.5 mg/mL. Reducing the pH allows the equilibrium to shift more to the lactone form and adversely impacts solubility. Dilute solutions of SDC-TRAP-0063 Sodium result in a lower pH with an increased risk of precipitation. The solubility of SDC-TRAP-0063 Sodium has also been seen to be adversely impacted by the use of 0.9% Sodium Chloride as a diluent. A common ion effect is the likely source of this observation. Non-ionic diluents, such as Mannitol, have been shown to provide higher solubility and are more suitable for the clinical dosing of SDC-TRAP-0063 Sodium.

A study was conducted with 0.6 mg/mL and 2.4 mg/mL solutions of SDC-TRAP-0063 Sodium using one of three diluents; 0.9% Sodium Chloride as a positive control, and 5% Dextrose and 5% Mannitol as potential new diluents (Table 2). Each diluent was prepared at the lower pH limit of USP to test a worst-case scenario for solubility (the starting pH was 4.5 for 0.9% Sodium Chloride and 5% Mannitol and 3.2 for 5% Dextrose). The SDC-TRAP-0063 Sodium dosing solutions were prepared in glass vials and then placed on a rocker. Appearance, pH and concentration of SDC-TRAP-0063 Sodium in the dosing solutions were analyzed by HPLC at 6 and 24 hours.

All three diluents show precipitation at a SDC-TRAP-0063 Sodium concentration of 0.6 mg/mL, with the highest recovery in 5% Mannitol at 6 hours. When solutions are prepared in the three diluents at a higher concentration of SDC-TRAP-0063 Sodium (2.4 mg/mL) the pH and solubility is seen to increase for all of the diluents, however precipitation is still noted in the 0.9% Sodium Chloride and 5% Dextrose solutions, whereas no precipitation and complete recovery was seen with 5% Mannitol.

TABLE 2
Particulate formation in SDC-TRAP-0063 Sodium dosing solutions using
Sodium Chloride, 5% Mannitol and 5% Dextrose adjusted to the low USP
limits (including rocking)

| | SDC-TRAP-0063 | | Recovery of SDC-TRAP-0063 | | |
|---|---|---|---|---|---|
| | Sodium | | Sodium (percent) and appearance | | |
| Diluent | Conc (mg/mL) | pH | 0 hours | 6 hours | 24 hours |
| 0.9% Sodium | 0.6 | 8.7 | 94.9% | 24.8% | 0.7% |
| Chloride, | | | Conforms¹ | Precipitate | Precipitate |
| pH 4.5 | 2.4 | 9.4 | 99.9% | 91.6% | 93.4% |
| | | | Conforms¹ | Precipitate | Precipitate |
| 5% Mannitol, | 0.6 | 8.6 | 99.1% | 62.8% | NT |
| pH 4.5 | | | Conforms¹ | Precipitate | Precipitate |
| | 2.4 | 9.8 | 100.3% | 99.4% | 99.8% |
| | | | Conforms¹ | Conforms¹ | Conforms¹ |
| 5% Dextrose, | 0.6 | 7.5 | 100.7% | 20.5% | NT |
| pH 3.2 | | | Conforms¹ | Precipitate | Precipitate |
| | 2.4 | 9.1 | 98.8% | 103.3% | 97.0% |
| | | | Conforms¹ | Conforms¹ | Precipitate |

¹Conforms = clear and essentially free of visible particles

The conclusion from this study is that there is a reduction in precipitation in SDC-TRAP-0063 Sodium dosing solution as the concentration of SDC-TRAP-0063 Sodium is increased with a concomitant increase in pH. Mannitol was selected as the diluent for further investigation because it demonstrated a superior stability profile when compared to either 0.9% Sodium Chloride or 5% Dextrose. In addition, the concentration of SDC-TRAP-0063 Sodium in Mannitol in subsequent studies including the in-use studies was increased to a minimum of 3 mg/mL to further mitigate the potential risk of precipitation.

Experiments were performed to assess the solubility of SDC-TRAP-0063 Sodium as a function of varying pH. Starting solutions with concentrations of 23.6 mg/mL in 5% Mannitol and 0.9% Sodium Chloride were equilibrated for period of approximately 24 hours after the addition of HCl and the concentration of the remaining SDC-TRAP-0063 Sodium solution was verified after filtering the mixture through a 0.2 μm filter.

The results are shown in Table 3 and Table 4. The control of the solubility and pH of the dosing solution was acceptable for Mannitol solutions and was found to be significantly worse for Sodium Chloride.

TABLE 3
Solubility of SDC-TRAP-0063 Sodium at various pH in 5% Mannitol

| | | SDC-TRAP- | SDC-TRAP-0063 | |
|---|---|---|---|---|
| Molar Percent | | 0063 Sodium | Sodium | |
| of HCl added | | concentration | Recovery (Percent | |
| to Mannitol | | in the solution | compared to initial | |
| solution | pH | (mg/mL)¹ | concentration) | Appearance |
| 0 | 10.1 | >23.6 (after 24 | 101.7% | Conforms² |
| | | hours of rocking) | | |
| 10.5 | 9.9 | >23.5 | 98.2% | |
| 18.2 | 9.9 | >23.3 | 101.7% | |
| 33.1 | 9.1 | 20.2 | 91.6% | |
| 35.5 | 9.4 | 20.9 | 93.5% | |

¹A reduction in PEN-866 Sodium concentration is expected due to the dilution related to the addition of HCl
²Conforms = clear and essentially free of visible particles

TABLE 4
Solubility of SDC-TRAP-0063 Sodium at various pH in 0.9% Saline

| Molar Percent of | | SDC-TRAP-0063 | SDC-TRAP-0063 | |
|---|---|---|---|---|
| HCl added to | | Sodium concentration | Sodium Recovery | |
| Mannitol | | in the solution | (Percent compared to | |
| solution | pH | (mg/mL) | initial concentration) | Appearance |
| 0 | 9.9 | 22.9 | 100.5% | Conforms¹ |
| 8.1 | 9.6 | 23.4 | 107.7% | Slightly hazy solution |
| | | | | (precipitation) |
| 14.4 | 9.1 | 21.4 | 78.1% | Slightly hazy solution |
| | | | | (precipitation) |
| 33.1 | 8.7 | 18.6 | 86.7% | Slightly hazy solution |
| | | | | (precipitation). |
| | | | | Complete |
| | | | | gelation after 1 hour |
| 35.5 | 8.7 | 13.7 | 64.1% | Slightly hazy solution |
| | | | | (precipitation). |
| | | | | Complete |
| | | | | gelation after 1 hour |

¹Conforms = clear and essentially free of visible particles

The pH of SDC-TRAP-0063 dosing solution is controlled by the concentration of SDC-TRAP-0063 Sodium and the observed pH for all dosing solutions in 5% Mannitol never dropped below 9.4 when tested using SDC-TRAP-0063 Sodium concentrations from 3-12 mg/mL. The results shown in Table 3 confirm that the maximum concentration of SDC-TRAP-0063 Sodium of 12 mg/mL does not result in precipitation in 5% Mannitol.

### Head-to-Head In-Use Test

A head-to-head in-use test of the SDC-TRAP-0063 Sodium 0.9% Sodium Chloride and 5% Mannitol dosing solutions was conducted. This study was intended to directly compare the two based on the specific clinical use direction for each diluent. In this study, the respective IV bags, syringes and IV lines were subjected to a rocking motion during the hold periods to mimic a worst case scenario for agitation of the dosing solution.

The 0.9% Sodium Chloride dosing solution was tested in an IV bag and IV line and at 0.6 mg/mL of SDC-TRAP-0063 Sodium as used in the clinic during the product complaint, with a designed 4 hour hold period followed by a 2-hour simulated infusion using a peristaltic pump (Table 5). Precipitation was observed in both the IV bag and IV line during the hold period and led to termination of the simulated infusion within 1 hour of onset due to occlusion of the in-line filter as indicated by repeated pump alarms. The SDC-TRAP-0063 recovery data from this study are shown in Table 5. Consistent with the observation of precipitation, low recoveries were observed in the IV lines at the beginning of infusion and from the bulk solution collected before the infusion was terminated.

TABLE 5
Appearance and recovery results for 0.6 mg/mL SDC-TRAP-0063 Dosing solutions in
Sodium Chloride collected at various points

| | | Average Recovery of | |
|---|---|---|---|
| | | 0.6 mg/mL | |
| | | SDC-TRAP-0063 | |
| | Sample Collection/ | Sodium siding solutions | |
| Time | Observation point | in Sodium Chloride | Appearance |
| 0 hours | IV bag | 100% | Conforms¹ |
| 2 hours | | 97.5% | Slightly hazy solution |
| | | | (precipitation) |
| 4 hours | | 96.7% | Conforms¹ |
| | | | (collected after in line |
| | | | filter) |
| 0 hours | IV Administration | 36.7% | Conforms¹ |
| | set | | (collected after in line |
| | | | filter) |
| Less than 1 hour | Drip Chamber of the | Not tested | Hazy with fine floating |
| (infusion | IV Administration | | particulates |
| Terminated²) | set | | |
| Termination of | Bulk solution, at | 55.9% | Conforms¹ |
| study² | termination | | (collected after in line |
| | | | filter) |

¹Conforms = clear and essentially free of visible particles
²Study was terminated within 1 hour of the start of infusion due to occlusion of the filter

The 5% Mannitol dosing solution was tested in a syringe and IV line as designed for use in the clinic with the addition of continuous rocking to simulate a worst case for agitation of the samples (Table 6). This dosing solution was tested at the limits of intended concentrations of 3 mg/mL and 12 mg/mL of SDC-TRAP-0063 Sodium and with a 2 hour hold period in the IV bag, a 6 hour hold period in the syringe and IV line and a 2-hour simulated infusion to exceed the planned clinical timeframes. With 5% Mannitol, no precipitation was observed at any time point and complete recovery of SDC-TRAP-0063 was observed.

TABLE 6
Assay results for SDC-TRAP-0063 Dosing solutions in 5% Mannitol
collected up to 8 hours

| | | Recovery | |
|---|---|---|---|
| | | of SDC-TRAP-0063 | |
| | | Sodium (Calculated vs. | |
| | | Initial concentration) | |
| Time Point | Sample Collection | 3 mg/mL | 12 mg/mL |
| 0 hours | IV bag | 100.0% | 100.0% |
| 2 hours | IV bag | 102.5% | 103.9% |
| 0 hours | IV line | 97.2% | 103.6% |
| 6 hours | IV line (infusion start) | 93.5%¹ | 101.6% |
| 8 hours (6 + 2 infusion) | IV line (infusion end) | 98.7% | 104.3% |
| 8 hours (6 + 2 infusion) | Bulk solution | 98.0% | 105.4% |

¹Low recovery due to the low volume of SDC-TRAP-0063 Sodium initially in the syringe at the 3 mg/mL concentration. A complete sample was not obtained for testing.

These results clearly indicate the stability of the SDC-TRAP-0063 Sodium dosing solution in 5% Mannitol under conditions of clinical use with timeframes that exceed the corresponding clinical periods. In contrast, SDC-TRAP-0063 Sodium in 0.9% Sodium Chloride under conditions that mimic the clinical use of this dosing solution resulted in precipitation and occlusion of the IV line filter.

To avoid potential contacts of SDC-TRAP-0063 dosing solutions in 5% Mannitol with Sodium Chloride, Sodium Chloride solution is excluded as a diluent and in the flushing of the IV line. To avoid any contact of SDC-TRAP-0063 dosing solutions with Sodium Chloride solution during administration, a flush of a Central Venous Line with 5% Mannitol pre- and post-infusion is implemented.

### Example 4: Stability Study of SDC-TRAP-0063 in Mannitol

In this study, SDC-TRAP-0063 sodium was dissolved in 5% Mannitol within the concentration range of 3-12 mg/mL. The stabilities of the SDC-TRAP-0063/Mannitol solution in an infusion container and during administration via a syringe pump were studied.

### Materials and Methods

The following list of materials and supplies were used in this study: SDC-TRAP-0063 Na; sterile water for injection, USP (WFI); intravia container (Baxter); 5% Osmitrol Injection, USP (Mannitol) (Baxter); 60 mL syringe (BD); 10 mL syringe (BD); 2 mL syringe (Norm-Ject); 1 mL syringe (BD); 18g needle (BD); syringe pump (Smiths Medical); IV administration set (60 in. extension set with 0.2 micron filter) (Smiths Medical); 20 mL scintillation vial (Kimble); and 40 mL scintillation vial (Chemglass).

### Design and Procedures

Dosing solutions of 3, 6 and 12 mg/mL of SDC-TRAP-0063 Na were prepared in 250 mL Intravia mixing containers. Pre-determined numbers of vials of SDC-TRAP-0063 Na were re-constituted with WFI and SDC-TRAP-0063 Na was transferred into mixing containers containing 5% mannitol. The exact amounts of each component are listed in Table 7. Each preparation was performed in duplicate.

TABLE 7
Preparation of 3, 6 and 12 mg/mL dosing solutions of SDC-TRAP-0063 Na

| Target | Number of SDC- | Volume of | Volume of 5% | Dosing Solution |
|---|---|---|---|---|
| Concentration, | TRAP-0063 Na | Reconstituted SDC- | Mannitol USP, | Volume in the Mixing |
| mg/mL | Vials | TRAP-0063 Na, mL | mL | Container, mL |
| 3 | 1 | 2 | 33 | 35 |
| 6 | 3 | 6 | 46.5 | 52.5 |
| 12 | 6 | 12 | 40.5 | 52.5 |

An in-use test was performed to simulate the clinical infusion process. Freshly prepared dosing solutions were withdrawn from the Intravia containers with the specified syringe size and volume, plus an additional 2 mL for flushing the administration set. The volume of the IV administration set is 0.7 mL; the 2 mL flush volume was selected according to standard pharmacy practice. After the administration set was flushed, the syringe was placed on a syringe pump and held at room temperature for 4 hours followed by a 2-hour infusion process. For the 15 mg dose level the simulation infusion was for 1 hour, because the minimal infusion rate specified by clinic was not less than 5 mL/hour. Samples were collected from the mixing containers at TO and T2 hours and from the administration sets at TO and from the bulk container at the end of infusion, at T5 hours for 15 mg sample and at T6 hours for all other samples, as outlined in Table 8.

TABLE 8
Analytical sampling and testing for the In-Use stability of
SDC-TRAP-0063 Na dosing solution

| | | SDC-TRAP-0063 Na testing | | |
|---|---|---|---|---|
| | | | | Assay/ |
| Time | | | | Impurity |
| point | Sample collection site | Appearance | pH | by HPLC |
| 0 h | Mixing container | X | X | X |
| 2 h | Mixing container | X | Not tested | X |
| 0 h | Administration set | X | Not tested | X |
| 6 h | Administration set | Not tested | Not tested | X |
| 6 h | Bulk that has come | X | X | X |
| | out of the | | | |
| | administration set | | | |

X = tested

A flexible bracketing design, intended to cover possible syringe volumes from 10 to 60 mL of nominal volumes, possible variation of the syringe fill volumes from 10 to 75% of the nominal syringe volume and concentration ranges of the dosing solutions from 3 to 12 mg/ml was executed in this study. The maximum fill volume for each syringe is limited to 75% of the nominal volume.

TABLE 9
In-Use study design

| | SDC-TRAP- | | Volume of | | |
|---|---|---|---|---|---|
| SDC-TRAP- | 0063 Na | Target volume | Dosing Solution | | |
| 0063 Na dose, | concentration, | of Dosing | in the syringe, | Syringe | Syringe pump |
| mg | mg/mL | Solution, mL | mL | volume, mL | setting, mL/hour |
| 15 | 3 | 5 | 7 | 10 | 5 |
| 30 | 3 | 10 | 12 | 60 | 5 |
| 204 | 6 | 34 | 36 | | 17 |
| 480 | 12 | 40 | 42 | | 20 |

### Results

All test samples appeared as clear solutions essentially free of particles. The pH of the SDC-TRAP-0063 Na dosing solutions were within the expected range of about 9.4 to about 10.3. The HPLC assay values for the SDC-TRAP-0063 Na samples collected during this study did not show any trends and were within expected ranges. The total impurities for the SDC-TRAP-0063 Na samples collected over a 2-hour period of storage in the mixing containers and over a total of 6 hours of simulated hold and infusion time (a total of 5 hours for 15 mg dose) were within the acceptable ranges for the impurities in SDC-TRAP-0063 Na concentrate and did not show any noticeable trends. In the HPLC assay analysis, no peaks above the integration threshold were observed in the blank infusion runs using 5 or 40 mL of just 5% Mannitol Injection, USP.

Hence, this in-use stability study for SDC-TRAP-0063 Na dosing solutions in 5% Mannitol confirmed an acceptable stability profile for the concentration range of 3-12 mg/mL of SDC-TRAP-0063 Na and sufficient compatibility with the IV administration set and infusion syringes.

### Example 5: A Phase 112a, Open-Label, Multicenter Study to Assess the Safety, Tolerability, Pharmacokinetics, Pharmacodynamics, and Preliminary Anti-Tumor Activity of SDC-TRAP-0063 in Patients with Advanced Solid Malignancies

### Study Drug

### Storage

SDC-TRAP-0063 Sodium, Sterile Powder for Solution for Infusion is stored refrigerated at 2° C. to 8° C., away from light.

SDC-TRAP-0063 Sodium, Sterile Powder for Solution for Infusion, is a lyophilized, sodium carboxylate form of SDC-TRAP-0063 and has a molecular formula of C₄₉H₅₀N₇₀O₁₀Na and a molecular mass of 919.95 g/mol. Under physiological conditions, SDC-TRAP-0063 Sodium equilibrates with the active lactone form of SDC-TRAP-0063. The drug product is supplied in 10 mL type I glass vials, placed within cartons to protect from light during storage. Prior to administration, the lyophilized powder is reconstituted with Water for Injection and then is diluted in 5% Mannitol, USP to the target concentration, and infused IV through a central venous access line.

### Preparation and Administration

SDC-TRAP-0063 Sodium, Sterile Powder for Solution for Infusion is diluted with water for injection (WFI), further diluted with 5% Mannitol and infused IV through a central venous access line.

SDC-TRAP-0063 Sodium, Sterile Powder for Solution for Infusion, is supplied in 10 mL type I glass vials.

The reconstituted solution of SDC-TRAP-0063 Sodium has a pH of around 10.0. This solution is diluted to the target dose in 5% Mannitol, USP. The pH of the infusion solution depends on the concentration of SDC-TRAP-0063 Sodium in the diluted infusion solution. Across the dose ranges employed in the clinical study protocol, the volume of the diluted infusion solution administered will range from 50 to 500 mL, and the pH will range from 8.1 to 9.6. In order to reduce the potential risks of injection site pain and/or damage to the venous endothelium during IV administration, a central venous access line is used for administration of the diluted SDC-TRAP-0063 Sodium.

To avoid potential contacts of SDC-TRAP-0063 dosing solutions in 5% Mannitol with Sodium Chloride, the Investigator Brochure now describes the exclusion of Sodium Chloride solution as a diluent and in the flushing of the IV line. A special precaution was added Pharmacy Manual and Clinical Protocol to avoid any contact of SDC-TRAP-0063 dosing solutions with Sodium Chloride solution during administration by implementing a flush of a Central Venous Line with 5% Mannitol pre- and post-infusion.

Hereinafter, SDC-TRAP-0063 refers to SDC-TRAP-0063 Sodium wherever there is a reference to the administered drug product and the doses to be administered for human use.

Patients receive SDC-TRAP-0063 administered IV over 120 minutes. Patients may be pre-medicated with an H1 antagonist and/or an IV corticosteroid per institutional policy if infusion-related reactions are experienced. Patients may also be administered prophylactic antiemetic medications, as indicated.

The diluted solution is attached to an infusion set free of diethylhexyl-phthalate (DEHP) and containing a 0.2 micron filter. SDC-TRAP-0063 infusion begins within approximately 2 hours of dilution and the infusion administration is completed within 2 (and no more than 4) hours from initiation, such that the total time from preparation of the diluted solution to completion of administration does not exceed 6 hours. The diluted solution is not light sensitive over a duration of 6 hours and does not need to be protected from light during reconstitution, dilution and administration.

### Overall Study Design

This study is a first-in-human, open-label, Phase 1/2a study evaluating the safety, PK, PDc, and anti-tumor activity of SDC-TRAP-0063 in patients with Ewing sarcoma or rhabdomyosarcoma, small cell lung cancer (SCLC), triple negative breast cancer (TNBC), pancreatic adenocarcinoma, colorectal carcinoma (CRC), or gastric adenocarcinoma. The study is carried out in 2 stages: Phase 1 (dose escalation) and Phase 2a (disease-specific cohort expansion).

The overall study design is presented in the Table below.

| Phase 1 | | | | Phase 2a |
|---|---|---|---|---|
| Dose Escalation | | | | Expansion |
| Number of dose cohorts dependent | | | | Ewing Sarcoma and |
| on toxicity results as determined by | | | | rhabdomyosarcoma (n = 20) |
| Bayesian logistic regression model | | | | |
| (BLRM) with overdose control | | | | |
| (EWOC) | | | | |
| | | | Dose 4 etc. | Small Cell Lung Cancer (n = 20) |
| | | Dose 3 | | Triple Negative Breast Cancer (n = 20) |
| | Dose 2 | | | Pancreatic adenocarcinoma (n = 20) |
| Dose 1 | | | | Colorectal carcinoma (n = 20) |

Gastric adenocarcinoma (n = 20)

### Screening

Patients are screened for study eligibility within 14 days [and within 28 days for tumor assessments by computed tomography (CT) or magnetic resonance imaging (Mill)] before the first study drug dose.

### Study

Patients who are determined to be eligible, based on screening assessments, and who have provided written informed consent for the study, begin treatment in the study on cycle 1 day 1 (C1D1, baseline). A treatment cycle is 4 weeks in length. All patients receive SDC-TRAP-0063 administered IV on D1, D8, and D15 of each cycle; the SDC-TRAP-0063 dose received is dependent on the cohort/phase in which the patient is enrolled. During treatment, patients attend study center visits and have study evaluations performed on D1, D8, and D15 of each treatment cycle. (Except for C1D1, visits during treatment cycles have a 1-day window.) All study visits are conducted on an out-patient basis but may be conducted on an in-patient basis per institutional policy.

Safety is assessed during the study by documentation of adverse events (AEs), clinical laboratory tests, physical examination, neurological examination, vital sign measurements, electrocardiograms (ECGs), and Eastern Cooperative Oncology Group (ECOG) performance status (PS).

Serial blood samples for pharmacokinetics (PK) are collected from all patients.

During screening, all sites of disease are assessed by CT. If the anatomic region cannot be adequately imaged by CT, MRI may be used instead. Tumor measurements are repeated within 7 days prior to the first study drug dose in every other cycle, and at the end of treatment (EOT) visit. Repeat assessments should use the same radiographic methods as used at baseline. Disease response is assessed using RECIST guidelines, version 1.1 (Eisenhauer et al., European Journal of Cancer, 45 (2009), 228-247, referred to as Eisenhauer 2009 hereinafter). Patients who achieve a partial response (PR) or complete response (CR) by RECIST are to have repeat assessments performed approximately 6 weeks later (and no sooner than 4 weeks from the prior assessment) to confirm the response. Following the confirmatory assessment, the response assessment schedule resumes at intervals of every other cycle. After end of treatment, for patients with stable disease or response, RECIST measurements continue until documented disease progression.

An eye exam is performed by an ophthalmologist during Screening, after the first cycle, every 3 cycles thereafter (or earlier if symptomatic), and at the End of Treatment (EOT) visit. The exams should include visual acuity, visual fields, and ophthalmoscopy. Electroretinogram (ERG) or dark adaptation tests are to be performed as determined by the evaluating ophthalmologist on a case by case basis. If at any time during the study a patient reports visual disturbances, study treatment is to be interrupted until an ophthalmologist performs an eye exam. Eye exams during the treatment phase are to be conducted within one week prior to dosing of the next treatment cycle. Eye exams at EOT may be conducted within ±4 weeks.

### Starting Dose

The SDC-TRAP-0063 selected starting dose is 30 mg IV on D1, D8, and D15 of each 28-day cycle. ICH S9 recommends that a starting clinical dose for a first-in-human study should be either 1/10th of the STD₁₀ in rodent toxicity studies or ⅙th of the HNSTD in non-rodent toxicity studies. In SDC-TRAP-0063 GLP toxicology studies, effects were demonstrated to be dose-dependent and generally reversible and readily monitored, with rats being the more sensitive species.

Based on the findings in the repeat dose GLP toxicity study in rats, the dose of one-tenth the rat STD₁₀ gives a Human Equivalent Dose (HED) of 0.48 mg/kg informing the SDC-TRAP-0063 first-in-human starting dose (Table 10). The calculation based on the dog highest non-severe toxic dose (HNSTD) of 20 mg/kg determined in the repeat dose GLP toxicity study in dogs is shown in Table 11 and results in a higher HED of 1.9 mg/kg substantiating that rat is the more sensitive species. The human starting dose of 0.48 mg/kg is equivalent to 18 mg/m². Assuming an average human weight of 60 kg, the starting dose translates to 29 mg or using an average body surface area of 1.7 m², the starting dose is 30 mg. Hence, we have selected a starting dose level of 30 mg.

TABLE 10
Human Starting Dose Calculation

| | | | Calculation of Human Starting Dose^{c} | |
|---|---|---|---|---|
| | 1/10 Rat | Rat 1/10 | HED of 1/10 Rat | HED of 1/10 Rat |
| Rat STD₁₀ | STD₁₀ | STD₁₀ | STD₁₀ | STD₁₀ |
| mg/kg | mg/kg^{a} | mg/m^{2b} | mg/kg | mg/m² |
| 30 | 3.0 | 18 | 0.48 | 18 |

^{a}As the rat is the most sensitive species, starting dose was calculated from 1/10 the rat STD₁₀.
^{b}Body Surface Area (BSA) = mg/kg dose in rat multiplied by 6 to calculate mg/m₂ dose in rat.

The HED in mg/kg was calculated by dividing 1/10 the rat STD₁₀ dose (in mg/kg) by 6.2 (assuming a 60 kg human). The human BSA dose (mg/m₂) was calculated by multiplying the mg/kg human dose by 37=mg/m₂.

TABLE 11
HNSTD in Dog for Comparison to Rat STD10

| | | | Human Equivalent Dosec | |
|---|---|---|---|---|
| | | | HED of | HED |
| | ⅙ Dog | Dog ⅙ | ⅙ Dog | of ⅙ Dog |
| Dog HNSTD | HNSTD | HNSTD | HNSTD | HNSTD |
| mg/kg | mg/kg^{a} | mg/m^{2b} | mg/kg | mg/m² |
| 20 | 3.3 | 67 | 1.9 | 69 |

^{a}For dog, ⅙ the HNSTD is considered an appropriate starting dose in humans.
^{b}BSA = mg/kg dose in dog multiplied by 20 to calculate mg/m₂ dose in dog.

The HED in mg/kg was calculated by dividing ⅙ the do HNSTD dose (in mg/kg) by 1.8 (assuming a 60 kg human). The human BSA dose (mg/m₂) was calculated by multiplying the mg/kg human dose by 37=mg/m₂.

Exposures achieved in these toxicology studies exceed the levels anticipated to show anti-tumor activity, based on anti-tumor efficacy determined in mice. The minimum dose for anti-tumor efficacy in mice xenografts was determined to be 20 mg/kg with once weekly administration. A 20 mg/kg dose in mice, resulted in the AUCₜ of 112 μg·h/mL. This exposure is similar to that in rats at the STD₁₀ in the GLP toxicology study where the AUCₜ values determined were 107 and 68 μg·h/mL in males and female rats, respectively. The mouse exposure is lower that the exposure at the HNSTD in dogs, 181 and 183 μg·h/mL in male and female dogs, respectively. Using body surface area, the dog HNSTD for SDC-TRAP-0063 is 6.6-fold higher than the minimum dose for efficacy in the mouse while the rat STD₁₀ is 3 fold higher.

### Screening Phase

After provision of written informed consent for the study, screening assessments include a careful review of the patient's medical history, assessment of Eastern Cooperative Oncology Group (ECOG) performance status (PS), physical examination, neurological examination, electrocardiogram (ECG) and laboratory assessments, and computed tomography (CT) or magnetic resonance imaging (MM) of all sites of disease.

Screening assessments are performed within 14 days before the first study drug dose, with the exception of CT or MM studies which may be performed within 28 days before the first study drug dose.

Patients who are determined to be eligible based on screening assessments are enrolled in the study on Cycle 1 Day 1 (C1D1; baseline).

### Treatment Phase (Phase 1 and Phase 2a)

The safety, pharmacokinetics (PK), and anti-tumor activity of SDC-TRAP-0063 will be assessed in all patients.

Safety is assessed during the study by vital sign measurements, physical examinations, neurological examinations, ECOG PS, documentation of adverse events (AEs), clinical laboratory tests, and ECGs.

Serial blood samples for PK assessments will be collected from all patients.

Tumor response assessments will be performed using Response Evaluation Criteria in Solid Tumors (RECIST), version 1.1 (Eisenhauer 2009) approximately every 8 weeks (i.e., every other treatment cycle, in which cycles are 4 weeks in duration). For patients who have a tumor response (complete or partial RECIST), a repeat evaluation to confirm response will be performed approximately 4 weeks after the initial response, i.e., after 1 additional treatment cycle.

During Phase 1 only, patients may undergo optional paired tumor biopsies and hair follicle collection for pharmacodynamic (PDc) assessment of the effect of SDC-TRAP-0063.

Patients may continue to receive SDC-TRAP-0063 as long as they are considered to show clinical benefit, and in the absence of meeting the discontinuation criteria.

### Phase 1 (Dose Escalation)

### Objectives:

### Primary

The primary objective of Phase 1 is to: Determine the MTD, select a RP2D, and generally investigate the safety and tolerability of SDC-TRAP-0063 when administered IV on days 1 (D1), 8 (D8), and 15 (D15) of 4-week treatment cycles in patients with advanced solid malignancies.

### Secondary

The secondary objectives of Phase 1 are to: Characterize the safety and tolerability of SDC-TRAP-0063, including both acute and chronic toxicities; Characterize the PK of SDC-TRAP-0063 and its components (HSP90 targeting ligand and SN-38), when administered IV in patients with advanced solid malignancies; Assess preliminary anti-tumor activity of SDC-TRAP-0063 in patients with advanced solid malignancies, using tumor response criteria as defined by RECIST 1.1, and duration of response.

### Exploratory

The exploratory objectives of Phase 1 are to: Assess preliminary anti-tumor activity of SDC-TRAP-0063 in patients with advanced solid malignancies by evaluating progression-free survival, overall survival, and tumor PDc biomarker changes as measured by γ-H2AX levels in patients' tumors approximately one week after administration of SDC-TRAP-0063; Explore the relationships between PK, efficacy, safety, and tumor PDc biomarker changes as measured by γ-H2AX levels in patients' tumors after administration of SDC-TRAP-0063; Explore the relationship between known tumor genomic or proteomic alterations identified in patients' tumors prior to treatment and anti-tumor activity of SDC-TRAP-0063; Explore the relationship between HSP90 levels in patients' tumors prior to treatment and anti-tumor activity of SDC-TRAP-0063.

Phase 1 employs an adaptive Bayesian logistic regression model (BLRM) with 2 parameters guided by the escalation with overdose control (EWOC) principle to make dose recommendations and estimate the maximum tolerated dose (MTD).

Patients receive SDC-TRAP-0063 administered intravenously (IV) over 2 hours in escalating dose cohorts on a 3 weeks on/1 week off schedule, i.e., treatment on days 1, 8, and 15 of each 28-day treatment cycle.

The starting dose of SDC-TRAP-0063 is 30 mg in the first dose cohort. To minimize the number of patients treated at potentially subtherapeutic dose levels, the first two dose cohorts enroll a minimum of 1 and no more than 2 patients, whereas subsequent cohorts will enroll a minimum of 3 patients.

Patients with a UGT1A1*28/*28 genotype identified during screening are not eligible to participate in Phase 1.

In the first 2 escalation cohorts, at least 1 patient must have completed Cycle 1 (C1) and have been assessed for safety and DLT for at least 4 weeks (including C2D1 pre-dose assessments) before enrollment of the next cohort may begin. In each dose escalation cohort following the second cohort, a minimum of 3 patients within a cohort are required to have completed C1 and have been assessed for safety and DLT for at least 4 weeks (including C2D1 pre-dose assessments) before enrollment of the next cohort may begin.

Statistical BLRM modeling is performed using all safety data and guide the selection of dose levels to be tested. In addition, PK and PD data may be used to inform dose selection. Dose escalation continues until the MTD is determined.

If during dose escalation SDC-TRAP-0063 related toxicities develop after administration of SDC-TRAP-0063 that delay administration of SDC-TRAP-0063 on day 8, day 8 dosing may be eliminated and continue dose escalation on an every 2 week dosing schedule, i.e., on days 1 and 15 of each 28 day treatment cycle, until the MTD on this alternate schedule is reached.

During Phase 1, if a patient is tolerating SDC-TRAP-0063 without significant evidence of disease progression, the patient may, beginning with C3 or subsequent cycle, have the dose increased to a dose that has already been established as tolerable. The dose may be increased only once for each patient.

The starting dose of SDC-TRAP-0063 is 30 mg. The planned dose levels are summarized in Table 12.

TABLE 12
Planned SDC-TRAP-0063 Dose Levels

| | | SDC-TRAP-0063 |
|---|---|---|
| Dose Level | % Increment from Prior Dose Level | Dose (mg) |
| −1 | (50% decrease) | 15 |
| 1 | Starting dose | 30 |
| 2 | 100% | 60 |
| 3 | 100% | 120 |
| 4 | 67% | 200 |
| 5 | 50% | 300 |
| 6 | 33% | 400 |
| 7 | 25% | 500 |
| 8 | 25% | 625 |
| 9 | 25% | 780 |

Actual dose increments may change but does not exceed a doubling of dose from the prior dose level. The doses assigned are guided by the updated results of BLRM.

Each patient in a dose cohort must have received SDC-TRAP-0063 in C1 and completed follow-up safety evaluations through C2D1 to be evaluable for the assessment of dose limiting tox (DLT). Patients who discontinue from the study for reasons other than DLT before completing C1 are replaced.

If a DLT necessitates enrollment of additional patients into a cohort, all safety data for that cohort are reviewed after all patients have received SDC-TRAP-0063 in C1 and completed follow-up safety evaluations through the end of C1. Based on the interim evaluation of the safety and tolerability data of the previous dose level, it may also be decided that accrual to take place at an intermediate dose level.

Toxicities are to be graded using the National Cancer Institute (NCI) Common Terminology for Cancer Adverse Events (CTCAE), version 4.03.

Although decisions regarding dose escalation are made based on review of data from C1, safety data are also collected from all patients continuing treatment and this is reviewed periodically. Any detected cumulative toxicity may require later dose reductions or other action as appropriate, including further refinement of the recommended phase 2 dose (RP2D).

### Phase 2a (Expansion)

### Objectives

### Primary

The primary objective of Phase 2a is to: Assess the efficacy of SDC-TRAP-0063 as a single-agent when administered IV using tumor response criteria as defined by RECIST 1.1 and duration of response in the following tumor-specific cohorts of patients with advanced solid malignancies whose disease has progressed during or after treatment with 1 or more prior lines of anticancer therapies: o Patients with Ewing sarcoma or rhabdomyosarcoma (n=20); Patients with small cell lung cancer (SCLC) (n=20); Patients with triple negative breast cancer (TNBC) (n=20); Patients with pancreatic adenocarcinoma (n=20); Patients with colorectal carcinoma (CRC) (n=20); Patients with gastric adenocarcinoma (n=20).

### Secondary

The secondary objectives of Phase 2a are to: Evaluate progression-free survival and overall survival in the above tumor-specific cohorts of patients; Evaluate the safety and tolerability of SDC-TRAP-0063 administration in the above tumor-specific cohorts of patients; Characterize the PK of SDC-TRAP-0063 and its components (HSP90 targeting ligand and SN-38) in the above tumor-specific cohorts of patients.

### Exploratory

The exploratory objective of Phase 2a is to: Explore the relationships between PK, efficacy, and safety in the above tumor-specific cohorts of patients.

Phase 1 is concluded and Phase 2a begins, once all patients treated in Phase 1 have been assessed for safety through and including C2D1, and all safety data have been reviewed.

SDC-TRAP-0063 is evaluated using the recommended Phase 2 dose (RP2D) identified at the conclusion of Phase 1. The RP2D is based on the findings of the safety, tolerability, PK, and PDc profile of SDC-TRAP-0063 during Phase 1. The RP2D may be the same as or below the MTD.

Patients with a UGT1A1*28/*28 genotype identified during screening are eligible to participate in Phase 2a. These patients are dosed at 75% of the RP2D in the first cycle. Dose adjustments in subsequent cycles are based on individual patient's safety and tolerability.

A total of up to 120 patients are treated in up to 6 expansion cohorts, each consisting of patients with distinct subsets of advanced solid malignancies (n=20 each) to assess the early efficacy, safety and PK of SDC-TRAP-0063 in these distinct populations.

### Number of Patients:

### Phase 1

Approximately 30 patients are enrolled. One to 2 patients are treated at the first two dose levels. All subsequent cohorts treat 3 to 6 patients at each dose level. An adaptive BLRM guided by the EWOC principle is employed to make dose recommendations and estimate the MTD. Approximately 4 to 6 dose escalation cohorts are anticipated. The total number of patients enrolled is dependent upon the observed safety profile as well as the number of dose escalation cohorts required to achieve the MTD and establish the RP2D of SDC-TRAP-0063.

Each patient will participate in only 1 dose cohort which is defined as the starting dose cohort.

### Phase 2a

A total of up to 120 patients are enrolled as follows: cohort 1 (n=20), cohort 2 (n=20), cohort 3 (n=20), cohort 4 (n=20), cohort 5 (n=20), cohort 6 (n=20). These cohort sample sizes are considered sufficient to obtain an early assessment of efficacy of SDC-TRAP-0063 in advanced cancer patients with distinct tumor types.

### Diagnosis and Main Criteria for Inclusion:

All patients (both Phase 1 and Phase 2a) must meet all of the following criteria to be eligible to participate:

1. Provision and understanding of signed and dated, written informed consent prior to any mandatory study-specific procedures, sampling, or analysis.
2. Male or female aged≥18 years.

### 3. ECOG PS of 0-1.

4. Adequate organ function within 14 days before C1D1, defined as follows: Bone marrow: Absolute neutrophil count (ANC)≥1.5×10⁹/L, platelet count≥100×10⁹/L, and hemoglobin≥9 g/dl.
   Hepatic: total bilirubin≤1.5×the upper limit of normal (ULN) and alanine aminotransferase (ALT) and aspartate aminotransferase (AST)≤2.5×ULN.
   Renal: If serum creatinine concentration≥1.5×ULN, then estimated creatinine clearance must be ≥50 mL/min (Cockroft-Gault formula).
   Patients must also meet these criteria when assessed within the three days before C1D1 to remain eligible.
   5. Serum potassium, calcium, magnesium, and phosphorus within normal limits. If values are low on the initial screening assessment, supplements may be given and values repeated to confirm within normal limits.
   6. If a female of childbearing potential, negative serum pregnancy test within 3 days before C1D1. A female of childbearing potential must agree to true abstinence or the use of highly reliable, physician-approved birth control from 14 days before C1D1 through 3 months after the last study drug dose. Highly reliable birth control means 2 of the following: (1) established use of oral, injected, or implanted hormonal methods of contraception, (2) placement of an intrauterine device, (3) condom or occlusive cap (diaphragm or cervical vault cap with spermicidal gel, foam, film, cream, or vaginal suppository), (4) male sterilization with verified absence of sperm in ejaculate post-vasectomy.
   7. If male, is surgically sterile or agrees to use a condom from C1D1 through 3 months after the last study drug dose.

Patients in Phase 1 must meet the following additional criterion:

8. Histologically- or cytologically-confirmed advanced solid malignancy having progressed after one or more prior lines of anticancer therapy. In addition, patients must have no other standard of care therapies that are deemed appropriate for the treatment of their malignancy.

For patients in Phase 1 who provide written informed consent to undergo an optional tumor biopsy during the Screening phase and again during SDC-TRAP-0063 treatment, such patients must meet the following additional criterion before undergoing a biopsy procedure:

9. Patient must have at least one site of tumor that is accessible to biopsy and that is considered by the Investigator to be low risk and of sufficient size to undergo a biopsy procedure on two separate occasions.

Patients in Phase 2a must meet the following additional criterion:

10. Measurable disease per RECIST 1.1 (i.e., at least 1 measurable lesion≥20 mm by conventional techniques or ≥10 mm by spiral CT scan or MRI), with the last imaging performed within 28 days before C1D1.
11. Patients must have a disease history as listed below specific to their disease:
Ewing sarcoma or rhabdomyosarcoma: Patients with locally recurrent or metastatic Ewing sarcoma or rhabodmyosarcoma whose disease has progressed after having received 2 or more prior lines of chemotherapy;
SCLC: Patients with advanced SCLC whose disease has progressed after having received one or more prior lines of chemotherapy. Patients are ineligible if their disease progressed during or within 3 months of having received irinotecan or topotecan as monotherapy or as a component of their most recent line of therapy;
TNBC: Patients with locally recurrent or metastatic TNBC who have received at least 2 previous chemotherapy regimens, including both an anthracycline and a taxane, and whose disease has progressed after having received one or more prior lines of chemotherapy for locally recurrent or metastatic disease;
Pancreatic adenocarcinoma: Patients with locally recurrent or metastatic pancreatic cancer whose disease has progressed after having received one or more prior lines of chemotherapy, including those whose disease has progressed within 6 months of postoperative adjuvant chemotherapy. Patients are ineligible if their disease progressed during or within 3 months of having received irinotecan as monotherapy or as a component of their most recent line of therapy;
CRC: Patients with metastatic colorectal carcinoma whose disease has progressed after having received 2 or more prior lines of chemotherapy for metastatic disease. Patients are ineligible if their disease progressed during or within 3 months of having received irinotecan as monotherapy or as a component of their most recent line of therapy;
Gastric adenocarcinoma: Patients with locally recurrent or metastatic gastric adenocarcinoma whose disease has progressed after having received one or more prior lines of chemotherapy. Patients are ineligible if their disease progressed during or within 3 months of having received irinotecan as monotherapy or as a component of their most recent line of therapy.

Patients meeting any of the following criteria are not eligible for study participation:

1. Treatment with anticancer therapy or an investigational drug within 2 weeks (6 weeks for mitomycin C and nitrosoureas), or within 5 half-lives of the agent if half-life is known and it is shorter, before C1D1. Anticancer therapies include cytotoxic chemotherapy, targeted inhibitors, immunotherapies, and radiotherapy, but do not include hormonal therapy. In addition, any drug-related toxicity, with the exception of alopecia and peripheral neuropathy, must have recovered to ≤Grade 1 (NCI CTCAE version 4.03).
2. Any other malignancy known to be active, with the exception of treated cervical intra-epithelial neoplasia and non-melanoma skin cancer.
3. One or more of the following cardiac criteria: Unstable angina; Myocardial infarction within 6 months prior to screening; New York Heart Association Class II-IV heart failure; Corrected QT interval (QTc)>470 msec obtained as the mean from 3 consecutive resting ECGs using the Fredericia formula; Clinically important abnormalities in rhythm, conduction, or morphology of resting ECG (e.g., complete left bundle branch block, third degree heart block); Congenital long QT syndrome; Symptomatic orthostatic hypotension within 6 months prior to screening;
Uncontrolled hypertension.
4. Stroke or transient ischemic attack within 6 month prior to screening.
5. Grade>2 peripheral neuropathy.
6. Patient requires medication with any of the inhibitors of UGT1A1, substrates of CYP1A2 or substrates of the P-glycoprotein (P-gp), breast cancer resistant protein (BCRP), organic anion uptake transporter polypeptide 1B1 and 1B3 (OATP1B1 or OATP1B3), or organic cation transporter 1 (OCT1) transporters. Patients receiving these drugs must undergo a two-week washout prior to C1D1.
7. History of leptomeningeal disease or spinal cord compression.
8. Brain metastases unless asymptomatic and not requiring steroids for at least 4 weeks prior to start of study treatment.
9. Major surgery within 28 days prior to C1D1.
10. If female, pregnant or breast-feeding.
11. As judged by Investigator, evidence of severe or uncontrolled systemic disease, active bleeding diatheses, renal or liver transplant, or active infection including known hepatitis B, hepatitis C, or human immunodeficiency virus (HIV).
12. Hypersensitivity or history of anaphylactic reaction to ganetespib or other HSP90 inhibitors.
13. Hypersensitivity or history of anaphylactic reaction to irinotecan, SN-38, or other agents containing irinotecan, SN-38 or its derivatives.
14. Any medical, psychological, or social condition that would interfere with the patient's participation in the study.

Patients in Phase 1 meeting the following additional criterion are not eligible for participation: 15. Genotype of UGT1A1*28/*28.

### Anti-Tumor Activity:

Disease response is assessed using RECIST 1.1. During Phase 1 only, for patients who undergo optional tumor biopsies and hair follicle collections prior to and during treatment with SDC-TRAP-0063, tumor PDc activity is assessed by measuring levels of γ-H2AX, a marker of DNA damage, in tumor tissue and hair follicle. All patients will be followed for progression-free survival and overall survival.

### Pharmacokinetics:

The PK profile is assessed by determining plasma levels of SDC-TRAP-0063 and its components (HSP90 targeting ligand and SN-38) from SDC-TRAP-0063 at intervals throughout the study.

### Statistical Methods and Data Analysis:

Data are summarized using descriptive statistics (continuous data) and/or contingency tables (categorical data) for demographic and baseline characteristics, efficacy measurements, safety measurements, and all relevant PK and PDc measurements.

### Analysis Populations

The Full Analysis set comprises all patients who receive any amount of SDC-TRAP-0063. The Safety Analysis set comprises all patients who receive any amount of study drug and have at least 1 post-baseline safety evaluation. The Dose Determining set comprises all patients who receive any amount of study drug and either experienced a DLT or have been followed for the full DLT evaluation period. The PK Analysis set comprises all patients who receive any amount of study drug and provide adequate PK samples. Patients with major protocol violations will be assessed on a patient-by-patient basis for inclusion in the PK Analysis set.

### Biomarker and Pharmacodynamic Assessments

In cell line panels, high expression of Schlafen-11 has shown a high positive correlation with response to topoisomerase I inhibitors and other DNA-damaging agents; and mutations in the Fanconi anemia gene FANCP (SLX4/BTBD12) has shown correlation with sensitivity to camptothecin and several other agents. Hence, patients' tumors collected prior to treatment with SDC-TRAP-0063 are analyzed retrospectively across a panel of known molecular genomic and/or proteomic alterations associated with human cancers and include mutations of FANCP and protein levels of Schlafen-11 to explore retrospectively whether there is an association between these alterations and patients' response to SDC-TRAP-0063.

In addition, HSP90α and HSP90β are differentially expressed at higher levels in tumors compared to normal cells. To explore whether HSP90 levels in patients' tumors might be associated with response to SDC-TRAP-0063, HSP90α and HSP90β are measured in patients' tumor samples prior to SDC-TRAP-0063 treatment.

The histone H2A variant H2AX (γ-H2AX) is an indicator of DNA double-strand breaks and is a sensitive marker of DNA damage response. During the DNA damage response, double-stranded breaks (DSB) are generated that result in the rapid phosphorylation of γ-H2AX. SDC-TRAP-0063 treatment of tumor cells induces DNA DSBs, characteristic of topoisomerase I inhibitors, such as the SDC-TRAP-0063 payload, SN-38. These DSBs can be quantitated by measuring γ-H2AX levels. In in vivo pharmacology studies, PDc response to SDC-TRAP-0063 was assessed by immunostaining for γ-H2AX and was associated with anti-tumor activity of SDC-TRAP-0063. γ-H2AX has been used in clinical studies to monitor DNA damage induced by topoisomerase I inhibition, ionizing radiation, or Wee1 inhibition. During Phase 1 only, for patients who sign the provision of optional tumor biopsies and hair follicle collections, levels of γ-H2AX are evaluated in paired tumor biopsies and hair follicles to check for evidence of SDC-TRAP-0063-induced DNA damage response. Based on the mechanism of action of SDC-TRAP-0063, it would be expected that evidence of DNA damage, as measured by γ-H2AX and/or other markers of DNA damage, would be greater in tumor tissue compared to hair follicle. It is estimated that 10 high quality pairs of tumor biopsies and hair follicles are needed to adequately explore the relationship between SDC-TRAP-0063 treatment and DNA damage response.

The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

We claim:

1. A method of treating cancer in a subject comprising administering an effective amount of SDC-TRAP-0063 sodium or a pharmaceutically acceptable salt thereof to the subject at a dose of at least about 0.48 mg/kg body weight or 18 mg/m² body surface area.

2. The method of claim 1, wherein SDC-TRAP-0063 sodium is dosed at least about 30 mg.

3. The method of claim 1, wherein SDC-TRAP-0063 sodium is dosed at less than about 800 mg.

4. The method of claim 1, wherein SDC-TRAP-0063 sodium is administered intravenously (IV).

5. The method of claim 4, wherein SDC-TRAP-0063 sodium is in a 5% Mannitol solution.

6. The method of claim 1, wherein SDC-TRAP-0063 sodium is administered once a week for 3 weeks on Day 1, Day 8, and Day 15.

7. The method of claim 6, wherein SDC-TRAP-0063 sodium is administered once a week for 3 weeks on Day 1, Day 8, and Day 15 followed with one week of no treatment.

8. The method of claim 7, wherein the 3-week on 1-week off treatment cycle of SDC-TRAP-0063 sodium is repeated for 8 weeks, 12 weeks, 16 weeks, 20 weeks, 24 weeks, 28 weeks, 32 weeks, 36 weeks, or 40 weeks.

9. The method of claim 1, wherein SDC-TRAP-0063 sodium is administered once every 2 weeks on Day 1 and Day 15.

10. The method of claim 9, wherein SDC-TRAP-0063 sodium is administered once every 2 weeks for 4 weeks, 8 weeks, 12 weeks, 16 weeks, 20 weeks, 24 weeks, 28 weeks, 32 weeks, 36 weeks, or 40 weeks.

11. The method of claim 1, wherein the cancer is selected from the group consisting of Ewing sarcoma or rhabdomyosarcoma, small cell lung cancer (SCLC), triple negative breast cancer (TNBC), pancreatic adenocarcinoma, colorectal carcinoma (CRC), and gastric adenocarcinoma.

12. A process of producing SDC-TRAP-0063 Sodium comprising the steps of:
1). dissolving SDC-TRAP-0063 in in a first portion of tert-butanol at 28-32° C.;
2). adding a second portion of tert-butanol;
3). adding 0.3 normal aqueous sodium hydroxide solution and Water for Injection to adjust pH to be above around 9.8;
4). filtering the mixture from step 3). with at least two 0.2 μm filters in series; and
5). conducting aseptic vial filling and lyophilization.

13. A pharmaceutical composition comprising an effective amount of SDC-TRAP-0063 Sodium, a tautomer thereof, or a pharmaceutically acceptable salt thereof, and 5% Mannitol.

14. The pharmaceutical composition of claim 13, wherein the pH is in the range of about 9.4 to about 10.3.

15. The pharmaceutical composition of claim 13, wherein the concentration of SDC-TRAP-0063 Sodium, a tautomer thereof, or a pharmaceutically acceptable salt thereof is in the range of around 1 mg/mL to around 20 mg/mL.

16. The pharmaceutical composition of claim 15, wherein the concentration of SDC-TRAP-0063 Sodium, a tautomer thereof, or a pharmaceutically acceptable salt thereof is about 3 mg/mL, 6 mg/mL, or 12 mg/mL.
